(19) United States
(12) Patent Application Publication
(10) Pub. No.: US 2024/0183863 A1
(43) Pub. Date: Jun. 6, 2024

(54) ELISA Assay for Measuring Function of Properdin and Kits for Conducting ELISA Assays using Anti-properdin Antibodies

(71) Applicant: The University of Toledo, Toledo, OH (US)

(72) Inventor: Viviana P. Ferreira, Toledo, OH (US)

(73) Assignee: The University of Toledo, Toledo, OH (US)

(21) Appl. No.: 18/410,121

(22) Filed: Jan. 11, 2024

Related U.S. Application Data
(63) Continuation-in-part of application No. 16/408,621, filed on May 10, 2019, now Pat. No. 11,913,960.
(60) Provisional application No. 62/670,314, filed on May 11, 2018.

(51) Int. Cl. G01N 33/68; G01N 1/40; G01N 33/543

(52) U.S. Cl. CPC G01N 33/6893; G01N 1/4077; G01N 33/54386; G01N 33/6854; G01N 2001/4088; G01N 2333/4716; G01N 2333/908

(57) ABSTRACT

Methods measuring properdin function and kits for conducting ELISA assays using anti-properdin antibodies and uses thereof are described.

### CROSS-REFERENCE TO RELATED APPLICATIONS

This is a continuation-in-part of U.S. application Ser. No. 16/408,621 filed under 35 U.S.C. § 111(a) on May 10, 2019, now allowed; which claims priority to U.S. Provisional Application No. 62/670,314 filed under 35 U.S.C. § 111(b) on May 11, 2018. The entire disclosures of all the aforementioned applications are expressly incorporated herein by reference for all purposes.

### STATEMENT REGARDING FEDERALLY SPONSORED RESEARCH

This invention was made with government support under Grant No. HL112937 awarded by the National Institutes of Health. The government has certain rights in the invention.

### BACKGROUND OF THE INVENTION

The complement system is a well-known effector mechanism of the immune response, providing not only protection against pathogens and other harmful agents but also recovery from injury. The complement system comprises a number of proteins that typically exist in the body in inactive form. In the complement system, pathogens are cleared from the circulation either by direct killing or when they bind to complement receptors on macrophages and other immune cells. Without the complement system, the pathogens would not be eliminated effectively. In addition, complement is important for normal and pathological inflammatory reactions. Complement can be activated through 3 pathways: the classical, lectin, and alternative pathways. The alternative pathway, unlike the other pathways, will activate on any surface that cannot control its activation, allowing C3 fragments to be deposited on pathogens (that cannot regulate the pathway), which will be either directly killed by complement or efficiently killed by immune cells that have bound to the C3 fragments. The alternative pathway can also activate on our own cells and tissues when they are not normal (such as dead or dying) as a physiological way of tagging them for removal by immune cells. Regulation of the alternative pathway is essential for its efficient function. There are proteins that negatively regulate to avoid unwanted damage to our own cells, and there is one positive regulator known as properdin that allows the alternative pathway to be very efficient. Failure in proteins of the alternative pathway will leave the individual more susceptible to infections and dying cells that, if not cleared, may serve as sources of altered self-antigens with the potential for inducing autoantibodies. In addition, excessive complement activation (e.g., due to massive tissue damage) or failure to adequately regulate complement activity (e.g., due to mutations) will result in exaggerated complement activity, with increased inflammation and disease susceptibility.

Complement deficiencies comprise between 1 and 10% of all primary immunodeficiencies (PIDs) according to national and supranational registries. However, there is a great lack of awareness among clinicians and general practitioners of such deficiencies.

Also, there are only a few centers worldwide that provide a comprehensive laboratory complement analysis. This is a concern as the genetic deficiency of properdin or of the terminal pathway components (C5 to C9) is highly susceptible to fulminant, systemic meningococcal disease.

Clinical indications for possible complement deficiencies include recurrent mild or serious bacterial infections, autoimmune disease, or episodes of angioedema (a painless, but often dramatic, swelling under the skin, or swelling in the intestines). Potential complement-related problems also include renal disease, vasculitis (blood vessel inflammation), atypicial hemolytic uremic syndrome, paroxysmal nocturnal hemoglobinuria, and age-related macular degeneration.

Currently there is no commercially available assay to measure properdin function in biological samples. Research labs (very specialized) have been limited to using functional assays requiring fresh red blood cells, carried out in 1-3 ml glass tubes. These assays are cumbersome because they require using animal cells that have a short shelf life. Furthermore, usually, the assay is carried out in 0.5 ml to 1 ml volumes, which uses high amounts of expensive reagents. In addition, there is no ELISA-based assay that measures properdin function.

### SUMMARY OF THE INVENTION

In a first aspect, there is provided herein an assay for determining the function of properdin, comprising: i) coating a surface with a non-inhibitory anti-properdin antibody, and incubating for a desired time and at a desired temperature; ii) adding a blocking agent to prevent non-specific binding and to block any remaining active sites on the surface, and incubating for a desired time and at a desired temperature; iii) adding a sample that contains properdin to the surface of step ii) and incubating for a desired time and at a desired temperature; iv) adding properdin-depleted serum to the surface of step iii); wherein the proper-depleted serum provides complement proteins including C3(H₂O), C3, Factor B, and Factor D, and, wherein C3(H₂O) and Factor B bind to properdin, and wherein Factor D cleaves Factor B to form C3(H₂O)Bb that, in turn, cleaves C3 and deposits C3b covalently on the proteins on the surface; and, v) adding an anti-C3b antibody to detect the C3b on the surface.

In another aspect, there is provided herein an enzyme linked immunosorbent assay (ELISA)-based method for detecting the function of properdin polymeric forms in normal and/or diseased biological samples, comprising the assay described herein.

In another aspect, there is provided herein a method for detecting nonphysiological forms of properdin (Pns) that appear during repeated freeze-thaw cycles while properdin is in storage, comprising: conducting the assay as described herein.

In certain embodiments, the sample comprises normal human serum (NHS) or serum from other species, or fluid samples from other parts of the body. In certain embodiments, the sample comprises a neutrophil supernatant. In particular embodiments, the neutrophil supernatant has a neutral pH. In certain embodiments, the sample comprises synovial fluid. In particular embodiments, the synovial fluid is from a subject having rheumatoid arthritis. In particular embodiments, the synovial fluid is from a subject having osteoarthritis. In certain embodiments, the sample comprises serum. In certain embodiments, the sample comprises ascitic fluid. In particular embodiments, the ascitic fluid is from a subject having a cancer.

In certain embodiments, the sample comprises atypical human serum, or serum from other species, or atypical samples for other parts of the body.

In certain embodiments, the assay is used in diagnostic applications.

In certain embodiments, the assay is used in research applications.

In certain embodiments, the non-inhibitory anti-properdin antibody comprises monoclonal antibody 6E9E6.

In certain embodiments, the control sample containing properdin is added at 800 ng/ml, and the test sample is added at an unknown concentration of properdin, and incubated at 37° C.

In certain embodiments, properdin-depleted sera is diluted 1/20 or 1/18-fold in 5 mM magnesium ethylene glycol tetraacetic acid (MgEGTA).

In certain embodiments, C3b deposition is measured by adding biotinylated anti-C3b antibody and streptavidin-horseradish peroxidase.

In another aspect, there is provided herein a method of detecting properdin function comprising: (i) obtaining a blood sample from a subject; (ii) detecting the presence of properdin in the blood sample by contacting the blood sample with non-inhibitory anti-properdin antibody; and (iii) detecting the binding of the non-inhibitory anti-properdin with a C3 antibody.

In another aspect, there is provided herein a method of diagnosing an alternative pathway (AP)-mediated pathology in a subject, comprising (i) conducting the assay described herein; (ii) determining the level of properdin function in the sample; and, (iii) treating the subject.

In certain embodiments, the subject has, or is suspected of having, a complement deficiency.

In certain embodiments, the subject has, or is suspected of having, a properdin deficiency.

In certain embodiments, the subject is a human subject.

In certain embodiments, a kit is provided that comprises a non-inhibitory anti-properdin antibody.

In certain embodiments, the kit additionally comprises reagents for an enzyme-linked immunosorbent assay (ELISA) test, selected from the group comprising slides, micro-titer plates, secondary antibodies, marker enzymes, and corresponding marker enzyme substrate for C3 antibody detection.

In another aspect, there is provided herein a kit for conducting an ELISA assay, comprising: at least one non-inhibitory antibody such as anti-properdin antibody (6E9E6), and at least one labelled anti-C3b antibody. In certain embodiments, the kit includes instructions.

Further provided is a method for conducting an enzyme linked immunosorbent assay (ELISA) for detecting the activity of properdin, the method comprising (i) coating a surface with a non-inhibitory anti-properdin antibody, and incubating for a desired time and at a first desired temperature; (ii) adding a blocking agent to prevent non-specific binding and to block any remaining active sites, and incubating for a second desired time and at a second desired temperature; (iii) adding a biological sample that contains properdin to the surface of step ii), and incubating for a third desired time and at a third desired temperature; and adding properdin-depleted serum to the surface of step iii), wherein the properdin-depleted serum provides complement proteins including C3(H₂O), Factor B, and Factor D, and, wherein C3(H₂O) and Factor B bind to properdin, and wherein Factor D cleaves Factor B to form C3(H₂O)Bb that, in turn, cleaves C3 and deposits C3b covalently on the proteins on the surface, and (v) adding an anti-C3b antibody to detect the C3b on the surface.

In certain embodiments, the biological sample comprises a neutrophil supernatant. In particular embodiments, the neutrophil supernatant has a neutral pH. In certain embodiments, the biological sample comprises synovial fluid. In particular embodiments, the synovial fluid is from a subject having rheumatoid arthritis. In particular embodiments, the synovial fluid is from a subject having osteoarthritis. In certain embodiments, the biological sample comprises serum. In certain embodiments, the biological sample comprises ascitic fluid. In particular embodiments, the ascitic fluid is from a subject having a cancer. In certain embodiments, the biological sample is added in ethylenediaminetetraacetic acid.

In certain embodiments, the method further comprises removing non-physiological forms of properdin (Pns) from the biological sample by size exclusion chromatography prior to adding the biological sample to the surface of step ii).

In certain embodiments, the non-inhibitory anti-properdin antibody comprises a monoclonal antibody. In particular embodiments, the monoclonal antibody is 6E9E6.

In certain embodiments, the properdin-depleted serum sample is diluted 1/20 or 1/18-fold in 5 mM magnesium ethylene glycol tetraacetic acid (MgEGTA).

In certain embodiments, C3b deposition is measured by adding biotinylated anti-C3b antibody and streptavidin-horseradish peroxidase.

Other systems, methods, features, and advantages of the present invention will be or will become apparent to one with skill in the art upon examination of the following drawings and detailed description. It is intended that all such additional systems, methods, features, and advantages be included within this description, be within the scope of the present invention, and be protected by the accompanying claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

PRIOR ART FIG. 1: Schematic illustration of a sandwich ELISA that detects the presence of properdin.

FIG. 2: Schematic illustration of a properdin functional ELISA. A plate is coated with a non-inhibitory anti-properdin MoAb (in red) and then incubated overnight at 4° C. The plate is blocked with PBS/3% BSA (in purple) to block the remaining active sites and incubated for 2 hours at 37° C. Properdin (light blue) is added and the plate is incubated for 1 hour at 37° C. Next, properdin depleted serum is added, which provides complement proteins including C3(H₂O) (green), Factor B, and Factor D (orange). C3(H₂O) and Factor B bind to properdin, Factor D cleaves Factor B to form C3(H₂O)Bb (de novo convertase formation on properdin) that can cleave C3 and deposit C3b (yellow) covalently on the proteins in the plate, and the C₃b is detected with a specific antibody (blue).

FIG. 3A: Physiological forms of properdin; dimers (P2), trimers (P3), and tetramers (P4) were separated from each other and from the nonphysiological aggregates (Pn) by size exclusion chromatography. The graph shows the O.D. values for fractions containing the properdin forms as labelled in FIG. 3A. The graph is a representative of 3 independent experiments.

FIG. 3B: Schematic illustration of properdin protein that exists in dimers (P2), left; trimers (P3), middle; and, tetramers (P4), right.

FIG. 4A: Functional ELISA with properdin forms and non-inhibitory anti-properdin MoAb 6E9E6. A 96 well plate was coated with 10 μg/ml of 6E9E6 and then incubated overnight at 4° C. The plate was then blocked with PBS/3% BSA for 2 hours at 37° C. Properdin forms were added at 800 ng/ml and plate was incubated for 1 hour at 37° C. Properdin-depleted sera was diluted 1/20 or 1/18-fold in 5 mM magnesium ethylene glycol tetraacetic acid (MgEGTA)-required for AP function, and added to the wells. The reaction was stopped at various times between 0-90 minutes, by adding 20 mM ethylenediaminetetraaceticacid (EDTA). C3b deposition was measured by adding biotinylated anti-C3b antibody and streptavidin-horseradish peroxidase. Finally, ABTS substrate (ABTS (2,2′-Azinobis [3-ethylbenzothiazoline-6-sulfonic acid]-diammonium salt) is a water-soluble HRP substrate that yields a green end product upon reaction with peroxidase) was added and the absorbance of each well at 405 nm was measured. The graph is representative of 4 independent experiments. Results are shown as mean and SD of duplicate observations.

FIG. 4B: C3b deposition at 50 minutes. This is the optimal time point for reading the assay. The significance of differences in C3b deposition between properdin forms at 50 minutes was assessed by one-way ANOVA with Tukey's multiple comparison test; p<0.0001 ****, p<0.001 ***, p<0.01 **, p>0.05 ns.

FIG. 5: The functional ELISA with properdin forms does not work if the anti-properdin MoAb is inhibitory (i.e., 6E11A4). The functional assay will not work if the anti-properdin antibody is inhibitory (i.e., inhibits properdin function). Thus, it is important for the assay to use a non-inhibitory anti-properdin antibody. A 96 well plate was coated with 10 μg/ml of 6E11A4 and then incubated overnight at 4° C. The plate was then blocked with PBS/3% BSA for 2 hours at 37° C. Properdin forms were added at 800 ng/ml and plate was incubated for 1 hour at 37° C. Properdin-depleted sera was diluted 1/20-fold in 5 mM MgEGTA (required for AP function) and added to the wells. The reaction was stopped at various times between 0-90 minutes, by adding 20 mM EDTA. C3b deposition was measured by adding biotinylated anti-C3b antibody and streptavidin-horseradish peroxidase. Finally, ABTS substrate was added and the absorbance of each well at 405 nm was measured. The experiment was performed once. Results are shown as single observations. No dose-dependent increase in C3b deposition is observed.

FIG. 6: Normal human serum (NHS) and properdin forms lead to a time-dependent deposition of C3b in the functional ELISA. A 96 well plate was coated with 10 μg/ml of 6E9E6 and then incubated overnight at 4° C. The plate was then blocked with PBS/3% BSA for 2 hours at 37° C. Properdin forms and NHS with 20 mM EDTA were added at 800 ng/ml and plate was incubated for 1 hour at 37° C. Properdin-depleted sera was diluted 1/18-fold in 5 mM MgEGTA (required for AP function). The reaction was stopped at various times 0-90 minutes, by adding 20 mM EDTA. C3b deposition was measured by adding biotinylated anti-C3b antibody and streptavidin-horseradish peroxidase. Finally, ABTS substrate was added and the absorbance of each well at 405 nm was measured. The graph is a representative of 3 separate experiments. Results are shown as single observations.

FIG. 7: The functional properdin ELISA detects differences in activity between sera that have different properdin concentrations. Assay was carried out as described in FIG. 6, except that the sera tested had varying amounts of properdin.

FIG. 8A: Properdin from feline serum is not recognized by the monoclonal antibodies.

FIG. 8B: Properdin from mouse serum is not recognized by the monoclonal antibodies.

FIG. 8C: Properdin from ferret serum is not recognized by the monoclonal antibodies.

FIG. 8D: Properdin from rat serum is not recognized by the monoclonal antibodies.

FIG. 9A: Properdin from baboon serum is recognized by the monoclonal antibodies.

FIG. 9B: Properdin from rabbit serum is recognized by the monoclonal antibodies.

FIG. 9C: Properdin from canine serum is recognized by the monoclonal antibodies.

FIG. 10: Visual representation of the method for determining the ratio of properdin oligomers using the properdin sandwich ELISA data from the fractions collected after performing size exclusion chromatography on NHS, from donor #4 as described in the materials and methods section in Example II herein. The width of the P4 peak was considered five fractions to the left of the P4 top fraction and all fractions to the right of the top fraction up until the fraction present in the valley between the P4 and P3. The fraction present at the valley between peaks was assigned to both P4 and P3 by dividing the concentration of that fraction equally between the two properdin oligomers. The width of the dimer peak was considered five fractions to the right of the P2 top fraction and all fractions to the left up until the valley between the P2 and P3 peaks. The fraction present at the valley between these peaks was assigned to both P2 and P3 by dividing the concentration of that fraction equally between the two properdin oligomers. The width of the P3 peak was defined as all fractions in between the P4/P3 valley and the P3/P2 valley. After assigning fractions to properdin oligomers, the concentration from fractions belonging to each properdin oligomer form was totaled. Then, all fractions, from all oligomers were summed for a grand total. The ratio of properdin oligomers was determined by dividing the total concentration of each properdin oligomer by the grand total and multiplying by 100.

FIG. 11: Principle of the properdin functional ELISA. An ELISA plate was coated with a non-inhibitory anti-properdin monoclonal antibody 6E9E6 (in green), blocked with BSA, and then properdin was captured from various sources (purified properdin, serum, neutrophil supernatant). The plate was washed, and properdin-depleted serum was added as a source of complement proteins including C3b, C3(H₂O), Factor B, and Factor D. C3b or C3 (H₂O) and Factor B bind to properdin to form C3 convertases, C3bBb, or C3(H₂O)Bb. Convertases cleave C3 and C3b is deposited covalently on BSA and detected with biotin-labeled anti-C3b (in red) followed by horseradish peroxidase-streptavidin, resulting in a colorimetric reaction.

FIGS. 12A-12B: Separation of properdin forms in purified properdin and NHS by size exclusion chromatography and quantification of properdin in the fractions by sandwich ELISA. Properdin from a pure, unfractionated properdin preparation (FIG. 12A) or in NHS (FIG. 12B) was separated into tetramers (P4), trimers (P3), dimers (P2), and non-physiological aggregates (Pn) by size exclusion chromatography, and the concentration of properdin in the collected fractions (diluted 1:3200 (A) and 1:100 (B)) was determined by sandwich ELISA as described in the materials and methods section of Example II herein and graphed.

FIGS. 13A-13C: Properdin functional ELISA can distinguish function between properdin oligomers derived from pure properdin and serum. Properdin function was quantified by the properdin functional ELISA where the readout was C3b deposition as described in the materials and methods section of Example II herein. Data is graphed minus background of samples containing no properdin during the properdin capture step. All samples were tested at equivalent concentrations (200 ng/ml). Properdin function was quantified in properdin oligomer samples (Pn, P4, P3, and P2) that were separated from pure properdin (A) and in P4, P3, and P2 from NHS (FIG. 13B). Both FIG. 13A and FIG. 13B include assessment of function of unfractionated NHS and unfractionated pure properdin as controls. The data is a representative of 3 experiments and is graphed as mean and standard deviation of triplicates. FIG. 13C shows C3b deposition normalized to an internal plate control (NHS, dotted line) for P2, P3, and P4 derived from pure properdin (as shown in FIG. 13A) and NHS (as shown in FIG. 13B). Significant differences in C3b deposition between samples was assessed by one-way ANOVA with Tukey's multiple comparison test; p<0.0001 ****, p<0.01 **, p<0.05 *, p>0.05 non-significant (only shown for FIG. 13C).

FIG. 14: Properdin functional assay detected dose-dependent increases in properdin function and wide functional differences between samples with distinct oligomeric composition. Properdin concentration of unfractionated pure properdin and of properdin in the context of NHS was determined by sandwich ELISA as described in the materials and methods section in Example II herein, followed by assessment of function in the samples using the properdin functional ELISA as described in the materials and methods section in Example II herein. Unfractionated pure properdin and properdin in NHS were assessed at varying properdin concentrations, as indicated in the x-axis. Data was graphed as mean and standard deviation of triplicates from one experiment.

FIG. 15: Properdin functional hemolytic assay does not detect greater function in higher order oligomers. Properdin function was quantified in unfractionated pure properdin and NHS, along with properdin oligomers separated from pure properdin (Pn, P4, P3, and P2), using a properdin functional hemolytic assay where % hemolysis is the read out as described in the materials and methods section of Example II herein. Data was graphed as mean and standard deviation of two independent experiments with duplicates.

FIG. 16: Properdin functional ELISA does not capture properdin in combination with C3 components. Properdin function was quantified by the properdin functional ELISA where the readout is C3b deposition as described in the materials and methods section of Example II herein. Data is graphed minus background of samples containing no properdin during the properdin capture step. All samples were tested at equivalent concentrations (200-300 ng/ml). Results are shown as mean and standard deviation of two independent experiments with triplicates. Significant differences in C3b deposition between samples was assessed by two-way ANOVA with Bonferroni post hoc test where p>0.05 non-significant (ns).

FIG. 17: Neutrophil-derived properdin had significantly lower function than serum-derived properdin, when assessed in the properdin functional ELISA, at equivalent concentrations. Properdin function was quantified by properdin functional ELISA where the readout was C3b deposition as described in the materials and methods section of Example II herein. Data is graphed minus background of samples containing no properdin during the properdin capture step. Properdin function in phorbol-12-myristate-13-acetate (PMA) activated neutrophil supernatant and serum collected from 10 healthy donors was quantified. All samples were tested at equivalent concentrations (300 ng/ml). Results are shown as mean and standard deviation from two independent experiments with triplicates. Significant differences in C3b deposition between samples was assessed by unpaired t test; p<0.0001 ****.

FIGS. 18A-18E: Properdin oligomeric distribution differed between neutrophil-derived and serum-derived properdin and was skewed towards dimers. The ratios of properdin oligomers in serum from donor #4 (FIG. 18A) and PMA-activated neutrophil supernatant from donor #4 (FIG. 18B), and PMA-activated neutrophil supernatant from donor #6 (FIG. 18C), #7 (FIG. 18D), #8 (FIG. 18E), #9 (FIG. 18F) were determined by size exclusion chromatography and properdin concentration by sandwich ELISA as described in the materials and methods section of Example II herein and the data was graphed by properdin concentration. The ratios of properdin oligomers were calculated as described in the materials and methods section of Example II herein and indicated as an inset to each graph.

FIG. 19: Properdin function does not differ in ovarian cancer or thrombotic diseases. Control sera (n=5, mean 1.3, SD 0.12) from patients who underwent surgery for a benign adnexal mass and was processed the same as ovarian cancer sera and ascites, sera from ovarian cancer patients (n=4, mean 1.3, SD 0.13), and ascites from ovarian cancer patients (n=5, mean=1.1, SD 0.12) and (B) plasma from healthy donors (n=5, mean 0.90, SD 0.15), patients with heparin-induced thrombocytopenia (HIT) (n=8, mean 084, SD 0.23), patients with thrombotic thrombocytopeni purpura (TTP) (n=9, mean 0.82, SD 0.25), and patients with active venous thrombosis at the time of blood draw (n=6, mean=1.0 SD 0.43) were assessed for properdin function by the properdin functional ELISA where the readout is C3b deposition as described in Example III herein. All samples were tested at equivalent properdin concentrations (300 ng/ml) with a properdin function reaction time of 90 min. Data is graphed as group mean and standard deviation each from triplicates. Statistical analysis determined by one-way ANOVA with Tukey's post hoc test for multiple comparisons.

FIG. 20: Properdin function was lower in osteoarthritis synovial fluid and serum compared to control serum. Control serum (n=20, mean 0.87, SD 0.30), osteoarthritis serum (n=18, mean 0.58, SD 0.22), and osteoarthritis synovial fluid (n=10, mean 0.61, SD 0.29) were assessed by properdin functional ELISA where the readout is C3b deposition as described in Example III herein. All samples were tested at an equivalent properdin concentration (100 ng/ml) with a properdin function reaction time of 90 min. Data is graphed as group mean and standard deviation. Statistical analysis determined by one-way ANOVA with Tukey's post hoc test for multiple comparisons. Non-significant results (p>0.05) are not shown.

FIG. 21: Properdin function was lower in rheumatoid arthritis synovial fluid compared to control serum. Control serum (n=20, mean 0.87, SD 0.30), rheumatoid arthritis serum (n=37, mean 0.74, SD 0.40), and rheumatoid arthritis synovial fluid (n=20, mean 0.57, SD 0.25) was assessed by properdin functional ELISA where the readout is C3b deposition as described in Example III herein. All samples were tested at an equivalent properdin concentration (100 ng/ml) with a properdin function reaction time of 90 min. Data is graphed as group mean and standard deviation. Statistical analysis determined by one-way ANOVA with Tukey's post hoc test for multiple comparisons. Non-significant results (p>0.05) are not shown.

### DETAILED DESCRIPTION

Before explaining at least one embodiment of the presently described inventive concept(s) in detail by way of exemplary drawings, description, experimentation, results, and/or laboratory procedures, it is to be understood that the inventive concept(s) is not limited in application to the details of construction and the arrangement of the methods, processes, compositions, and components set forth in the following description or illustrated in the drawings, experimentation and/or results. The inventive concept(s) is capable of other embodiments or of being practiced or carried out in various ways. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting except where indicated as such.

Unless defined otherwise, all technical and scientific terms used herein have the same meaning as commonly understood by one of ordinary skill in the art to which this invention belongs. Generally, the nomenclature used herein and the laboratory procedures are well known and commonly employed in the art. Conventional methods are used for these procedures, such as those provided in the art and various general references. Where a term is provided in the singular, the inventors also contemplate the plural of that term unless the context clearly indicates otherwise.

As used herein, the singular forms “a”, “an” and “the” include plural reference unless the context clearly dictates otherwise. Unless defined otherwise, all technical and scientific terms used herein have the same meanings as commonly understood by one of ordinary skill in the art. Although any methods similar or equivalent to those described herein can be used in the practice or testing of embodiments of the present invention, the preferred methods are now described. Nothing herein is to be construed as an admission that the invention is not entitled to antedate such disclosure by virtue of prior invention.

As used herein, the term “about” means plus or minus 10% of the numerical value of the number with which it is being used. Therefore, about 50% means in the range of 45%-55%.

As used herein, the terms “optional” or “optionally” may be taken to mean that the subsequently described structure, event or circumstance may or may not occur, and that the description includes instances where the event occurs and instances where it does not.

As used herein, the term “administering” when used in conjunction with a therapeutic means to administer a therapeutic directly into or onto a target or to administer a therapeutic to a subject whereby the therapeutic positively impacts the area to which it is targeted.

The term “detect” or “detecting” is used in the broadest sense to include quantitative, semi-quantitative or qualitative measurements of a target molecule. In one aspect, methods described herein may only determine the presence or absence of a particular properdin polypeptide in a biological sample and, thus, that the properdin polypeptide is detectable or, alternatively, undetectable in the sample when assayed by the method.

As used herein, the terms “treat,” “treating” or treatment” generally mean the exposure of a living organism to one or more physical, chemical, or psychological entities or stimuli that may prevent, improve, or ameliorate a diseased state. As used herein, the term “treating” refers to both therapeutic treatment and prophylactic or preventative measures.

As used herein, the term “indication” generally refers to a medical condition or symptoms associated with a medical condition.

As used herein, “subject” refers to any mammal, including humans, domestic and farm animals, and zoo, sports, or pet animals, such as dogs, horses, cats, sheep, pigs, cows, etc. The preferred mammal herein is a human, including adults, children, and the elderly.

As used herein, “preventing” means preventing in whole or in part, ameliorating, or controlling.

As used herein, a “therapeutically effective amount” in reference to the compounds or compositions refers to the amount sufficient to induce a sign or any other desired alteration that results in the promotion and/or improvement of a subject's health.

As use herein, the term “improves” generally means changes either the appearance, form, characteristics and/or the physical health.

### GENERAL DESCRIPTION

The complement system is a central component of the innate immune system. It participates in direct killing of pathogens, in opsonization and removal of pathogens and altered self cells (e.g., apoptotic, necrotic, etc.), and in generation of pro-inflammatory fragments that increase vascular permeability, have potent chemotactic abilities, and can activate phagocytic, pro-inflammatory cells). The complement system can be activated by three pathways (the classical, lectin, and alternative pathways).

Properdin is a highly positively charged 53 kDa glycoprotein that is the only known positive regulator of the alternative pathway of the complement system. Properdin stabilizes complement enzymes (the C3 and C5 convertases), such that their activity increases 5-10-fold, thus leading to efficient amplification of C3b deposition on the cell surfaces. Properdin is composed of seven thrombospondin repeat type I (TSR0-6) domains. Under physiological conditions properdin exists as cyclic dimers (P2), trimers (P3), and tetramers (P4) in a 26:54:20 (P2:P3:P4) ratio and the convertase stabilizing function of P4 is greater than P3 and P3 is greater than that of P2. Head-to-tail associations of four TSR domains from two monomers form a curly vertex that holds together alternative pathway (AP) convertase by interacting with both C3b and Bb. TSR4 is required for stabilizing the C3bBb convertase, TSR5 is important in C3b and sulphatide binding and, TSR6 is essential for properdin polymerization whereas TSR3 domain is not required for C3b stabilization or sulphatide binding.

In addition to its role as a regulator of the AP, properdin can also act as an initiator of the AP by selectively binding to target surfaces and providing a platform for de novo convertase assembly. Examples of such cell surfaces include zymosan, late apoptotic and necrotic cells, C. pneumoniae, and activated platelets.

Nonphysiological forms of properdin (Pns) appear during repeated freeze-thaw cycles while properdin is in storage and have distinctly different properties from physiological forms since they bind non-specifically to surfaces and consume complement, even in solution.

Thus, it is essential to separate the physiological forms from the Pns by ion exchange or size exclusion chromatography to accurately assess properdin function.

Also, in humans, deficiency of properdin leads to susceptibility to bacterial infections, especially to severe fulminant meningococcal infections. Complete deficiency of properdin leads to Type I deficiency, partial deficiency leads to Type II, and Type III is due to dysfunctional properdin.

Also, properdin is now believed to play an important role in inflammatory diseases such as thromboinflammation, as demonstrated by its role in enhancing platelet granulocyte aggregate formation in thrombin receptor-activated whole blood, as well as having a role in a variety of diseases where the activation of the alternative pathway is central to the pathogenesis (e.g., atypical hemolytic uremic syndrome, paroxysmal nocturnal hemoglobinuria, and many chronic inflammatory diseases).

In normal humans, the concentration of properdin in serum ranges from about 4 to about 25 μg/ml. Various leukocytes including mast cells, monocytes, dendritic cells, T cells, and neutrophils are known to produce properdin.

However, in local inflammatory microenvironments, properdin concentration may increase in response to leukocyte activation, causing inflammation due to amplification of the activity of the complement system.

Currently there is no in vitro enzyme linked immunosorbent assay (ELISA)-based method to detect the function of properdin polymeric forms in any normal or diseased biological sample.

Described herein is the use of anti-properdin monoclonal antibodies (MoAbs) in an assay that measures the function of properdin in any biological sample that contains properdin including, but not limited to, normal human serum (NHS), atypical human serum, neutrophil supernatants, synovial fluid, and ascitic fluid. The assay has applications in both diagnostics and research.

### EXAMPLES

Certain embodiments of the present invention are defined in the Examples herein. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

The present invention is further defined in the following Examples, in which all parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. All publications, including patents and non-patent literature, referred to in this specification are expressly incorporated by reference. The following examples are intended to illustrate certain preferred embodiments of the invention and should not be interpreted to limit the scope of the invention as defined in the claims, unless so specified.

The value of the present invention can thus be seen by reference to the Examples herein.

### Example I

### Standardization of a Properdin Functional ELISA

FIG. 1 is a general schematic illustration of a sandwich ELISA that detects properdin with maximum sensitivity. In order to be able to measure properdin levels in biological samples, a highly sensitive sandwich ELISA using anti-properdin monoclonal antibodies was standardized, where one monoclonal antibody has been biotinylated.

### Isolation of Properdin Forms

Physiological polymeric forms of properdin dimers (P2), trimers (P3) and tetramers (P4) were separated from non-physiological aggregated properdin (Pns) by size exclusion chromatography. Briefly, properdin (5 mg) was loaded onto a Phenomenex Bio Sep-Sec-S4000 column (600×7.8 mm) with a guard column (75×7.8 mm), and was eluted at a flow rate of 0.5 ml/min in PBS. Purified, physiological and non-physiological forms of properdin were stored at 40C and used within three weeks of separation.

### Standardization of the Functional ELISA Using Properdin Forms

In order to standardize the functional ELISA, a series of experiments was conducted where 96 well plates were coated with varying concentrations (1-20 g/ml) of non-inhibitory MoAbs 1G6D2 or 6E9E6 in PBS (100 μl/well) and then incubated overnight at 4° C.

“1G6D2” monoclonal antibody is available from HycultBiotech, under Catalog #: HM2354-FS.

“6E9E6” monoclonal antibody is available from Millipore Sigma, under Cat. No. MABF2125.

The plates were washed four times with PBS/0.05% Tween (250 μl/well). PBS/3% BSA (250 μl/well) was added to block remaining active sites and the plates were incubated for 2 hours at 37° C., followed by 2 washes with PBS/0.05% Tween (250 μl/well). Properdin forms [P2, P3, P4, or pooled P2-P4 at a 1:2:1 ratio as found in NHS, or Pn, or Punfrac (pure properdin with physiological P2-P4 polymers and non-physiological aggregates/unfractionated properdin)] were diluted in PBS/1% BSA/0.05% Tween and added at varying concentrations of 0-1000 ng/ml (100 μl/well). The plates were then incubated for 1 hour at 37° C. followed by 4 washes with PBS/0.05% Tween (250 μl/well).

Next, properdin-depleted sera in varying dilutions (1/10-1/1000-fold) was added in GVB=+5 mM MgEGTA (100 μl/well). GVB=+20 mM EDTA was added at varying time points between 0-90 minutes to stop the reaction followed by 4 washes with PBS/0.05% Tween (250 μl/well).

The C3b deposited covalently on the proteins in the plates was detected by incubating with varying concentrations (1/2000-1/10000) of biotinylated anti-C3b antibody (100 μl/well) at 37° C. for 1 hour followed by 4 washes with PBS/0.05% Tween (250 μl/well). Streptavidin-horseradish peroxidase at 1/5000 (100 μl/well) was added and incubated at 37° C. for 45 minutes followed by 4 washes with PBS/0.05% Tween (250 μl/well) and then ABTS substrate was added and the absorbance of each well was measured at 405 nm using a Tecan Infinite M200 spectrophotometer.

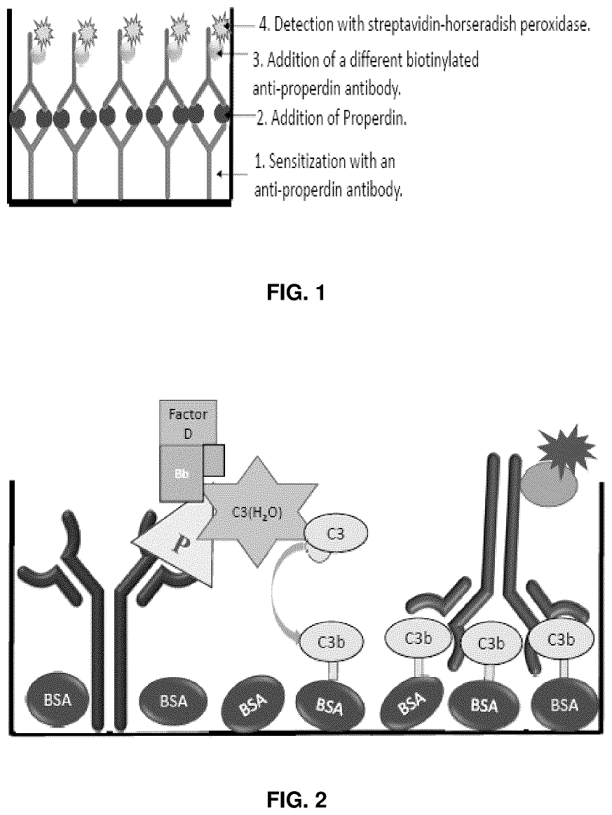

FIG. 2 is a schematic illustration showing a plate is coated with a non-inhibitory anti-properdin MoAb (in red); blocked with PBS/3% BSA (in purple) to block the remaining active; addition of properdin (light blue); addition of properdin depleted serum, which provides complement proteins including C3(H₂O) (green), Factor B, and Factor D (orange). C3(H₂O) and Factor B bind to properdin, Factor D cleaves Factor B to form C3(H₂O)Bb (de novo convertase formation on properdin) that can cleave C3 and deposit C3b (yellow) covalently on the proteins in the plate and the C3b is detected with a specific antibody (blue).

Evaluation of Properdin Forms in their Ability to Initiate C3b Deposition in a Functional ELISA

A 96 well plate was coated with 10 μg/ml 6E9E6 (non-inhibitory anti-properdin MoAb) or 6E11A4 (inhibitory anti-properdin MoAb) in PBS (100 μl/well) and then incubated overnight at 4° C. The plate was washed four times with PBS/0.05% Tween (250 μl/well). PBS/3% BSA (250 μl/well) was added to block the remaining active sites and the plate was incubated for 2 hours at 37° C., followed by 2 washes with PBS/0.05% Tween (250 μl/well). Properdin (P2, P3, P4, or pooled P2-P4 at a 1:2:1 ratio as found in NHS, or Pn, or Punfrac was diluted in PBS/1% BSA/0.05% Tween added at 800 ng/ml (100 μl/well). The plate was then incubated for 1 hour at 37° C. followed by 4 washes with PBS/0.05% Tween (250 μl/well).

Next, properdin-depleted sera (either 1/20 or 1/18, depending on the serum batch) was added in GVB=+5 mM MgEGTA, required for AP activity (100 μl/well). The reaction was stopped with GVB=+20 mM EDTA at 0-90 minutes followed by four washes with PBS/0.05% Tween (250 μl/well). The C3b deposited covalently on the proteins in the plate was detected by incubating with a biotinylated anti-C3b antibody 1/5000 (100 μl/well) at 37° C. for 1 hour followed by 4 washes with PBS/0.05% Tween (250 μl/well). Streptavidin-horseradish peroxidase at 1/5000 (100 μl/well) was added and incubated at 37° C. for 45 minutes followed by 4 washes with PBS/0.05% Tween (250 μl/well) and then ABTS substrate was added and the absorbance of each well was measured at 405 nm using a Tecan Infinite M200 spectrophotometer.

### Evaluation of Biological Samples in the Functional ELISA

### Determination of the Activity of Properdin in NHS Using Functional ELISA

The assay was carried out as described above, with the following exceptions: after the blocking step, properdin (P2, P3, P4, P2-P4 at a 1:2:1 ratio, Pn, Punfrac) at 800 ng/ml in PBS/1% BSA/0.05% Tween or NHS (Innovative Research or Comptech) at a dilution that provides 800 ng/ml of properdin diluted in 20 mM EDTA+PBS/1% BSA/0.05% Tween, was added at 100 μl/well. The NHS were added in 20 mM EDTA to avoid complement activation during this properdin recruitment step.

The plate was then incubated for 1 hour at 37° C. followed by 4 washes with PBS/0.05% Tween (250 μl/well). Next, 1/18 dilution of properdin-depleted sera was added in GVB=+5 mM MgEGTA, required for AP activity (100 μl/well).

The reaction was stopped with GVB=+20 mM EDTA at 0-120 minutes followed by four washes with PBS/0.05% Tween (250 μl/well).

### Results

### Standardization of a Properdin Functional ELISA

### Separation of Properdin Forms

Non-physiological aggregates of properdin (Pn) accumulate in purified properdin preparations, due to prolonged storage and freeze/thaw cycles, are artificially highly active and should be removed before use in research.

Properdin deficiency leads to increased susceptibility to meningitis and gonorrhea (systemic, recurrent Neisserial infections). In type III deficiency there is a normal concentration of protein, but without function.

The alternative pathway of complement participates in the pathogenesis of many inflammatory diseases (including cardiovascular and autoimmune, etc.), and properdin (positive AP regulator) function in disease settings is not fully understood.

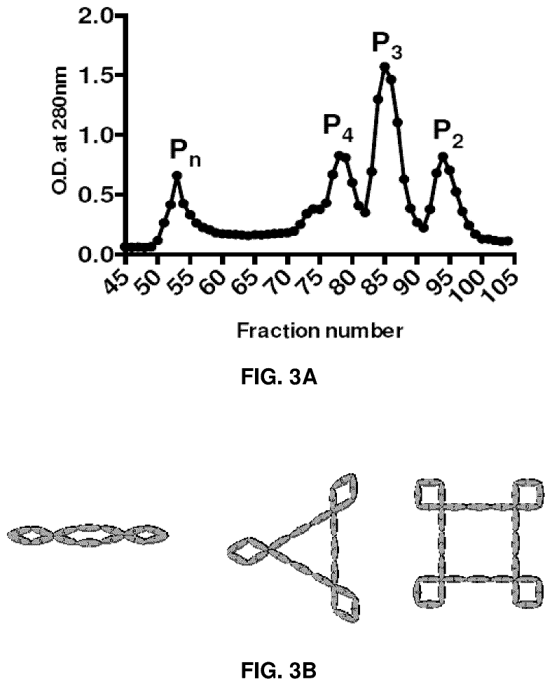

In order to be able to standardize the properdin functional ELISA using properdin forms, physiological forms of properdin dimers (P2), trimers (P3), and tetramers (P4) were separated from each other and from the non-physiological aggregates (Pns), that appear during repeated freeze-thaw cycles, by size exclusion chromatography (FIG. 3A). FIG. 3B provides a schematic illustration of properdin protein that exists in dimers (P2), left; trimers (P3), middle; and, tetramers (P4), right.

### Standardization of the Functional ELISA Using the Properdin Forms

In order to standardize a functional ELISA for properdin (see FIG. 2), preferred reagents and concentrations to use for the assay were determined:
(a) which non-inhibitory MoAb is useful to use for capturing the properdin from samples and what is a preferred concentration would be;
(b) what concentration of properdin would give a preferred functional signal;
(c) what dilution of properdin-depleted serum is preferred;
(d) what is the preferred concentration of biotinylated anti-C3b antibody to use; and
(e) what is a preferred time range that allows to detect functional differences between the properdin forms.

A series of standardization experiments were performed where all the above-mentioned parameters were varied and the data indicated that:
(a) the MoAb that detected the most differences between properdin forms function was 6E9E6 at 10 μg/ml;
(b) the function of properdin (i.e., C3b deposition) is consistently detected with 800 ng/ml of properdin;
(c) the dilution of properdin-depleted serum is useful at 1/20 or 1/18 depending on the batch of serum used;
(d) the concentration of biotinylated anti-C3b antibody dilution is useful at 1/5000; and
(e) the time points where properdin function could be consistently detected was 0-90 min. Results for the basic standardization are not shown.

In order to standardize the functional ELISA, varying the concentration of the sensitization antibody, properdin, the dilution of p-depleted sera, and the dilution of anti-c3b antibody were varied, as shown in Table 1 below. All these parameters were measured at varying timepoints. Example 1 shows one embodiment that uses the sensitization antibody at 10 μg/ml, properdin between 300 and 800 ng/ml, p-depleted sera at 1/20 or 1/18 fold dilution depending on sera lot, and 1/5000 dilution of anti-c3b antibody.

TABLE 1

| | Concentrations/ | Example 1 = |
|---|---|---|
| Steps of the | dilution/ | Concentrations/ |
| functional ELISA | time range | dilutions/time range |
| Sensitization antibody | 1-20 μg/ml | 10 μg/ml |
| Properdin | 0-1000 ng/ml | 400-800 ng/ml |
| Properdin- | 1/10-1/1000-fold | 1/18 or 1/20-fold |
| depleted sera | | |
| Biotinylated anti- | 1/2000-1/10000 | 1/5000 |
| C3b antibody | | |
| Time range | 0-90 minutes | 0-90 minutes |

The protocol for the final standardized properdin functional assay as well as data for the differences in function between the properdin forms, as detected in the functional assay, are described below.

Evaluation of Properdin Forms in their Ability to Initiate C3b Deposition in a Functional ELISA

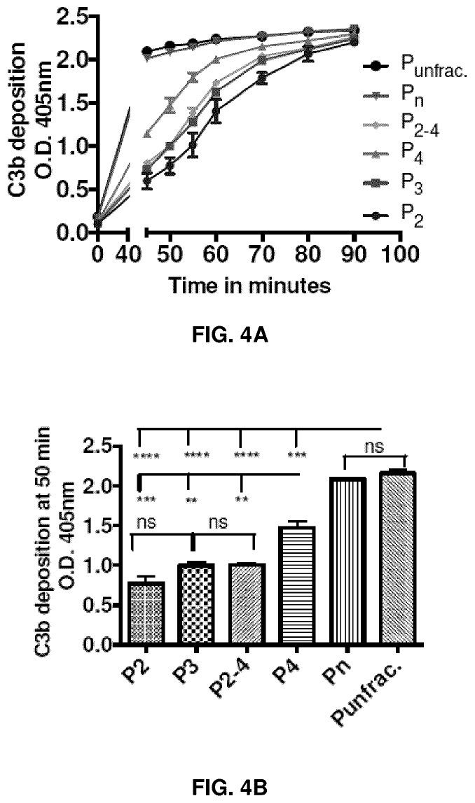

In order to validate the functional ELISA, different properdin forms were tested (which exhibit differences in their ability to promote complement activation (Pn>P4>P3>P2). The functional ELISA was performed using the non-inhibitory anti-properdin MoAb 6E9E6 (see method schematic in FIG. 2), and the function of the different properdin forms was measured at various time points between 0-90 minutes (FIG. 4A).

The data shows that all properdin sources tested led to deposition of C3b in a time-dependent manner. Between 45-55 minutes Punfrac (which is pure properdin that contains physiological P2-P4 polymers and non-physiological aggregates), and Pn had significantly higher activity than all the physiological forms of properdin (FIG. 4A). In addition, P4 showed a significantly higher activity than P3 and P2 (FIG. 4B).

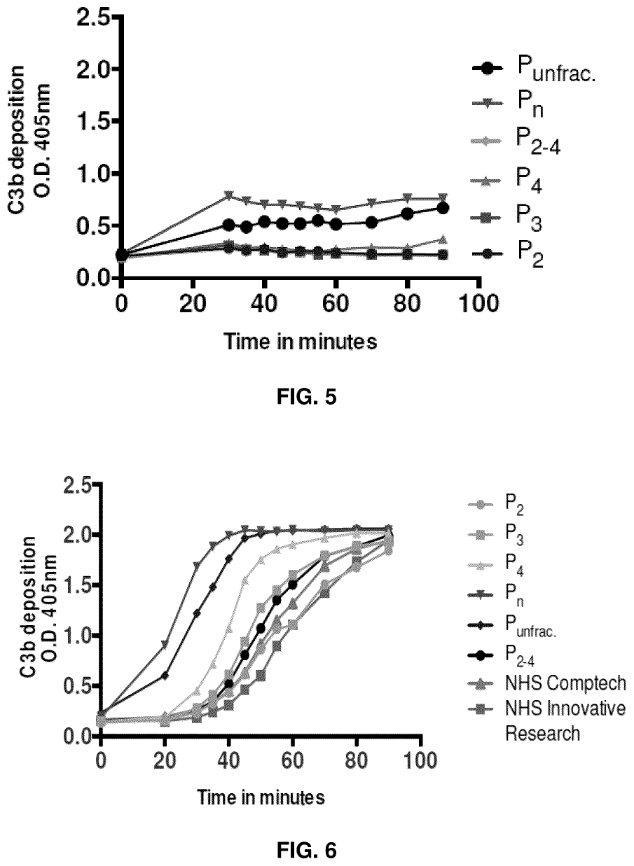

The inhibitory anti-properdin MoAb 6E11A4 could no longer detect the differences between the functions of the physiological forms of properdin (FIG. 5); while not wishing to be bound by theory, it is now believed that this is due to the functional inhibition of the captured properdin, although, there was some C3b deposition with the Pn and Punfrac.

### Evaluation of Biological Samples in the Functional ELISA

Functional ELISA with NHS

The function of properdin in the context of NHS was evaluated in parallel with different properdin forms (individual properdin forms, P2-P4 pool, and Pn) and with Punfrac. All samples were tested at an equivalent concentration of 800 ng/ml. When the function of properdin was tested between 0 and 90 minutes, NHS from two sources and all the properdin forms tested resulted in deposition of C3b in a time-dependent manner (FIG. 6). The C3b deposition with the NHS was more similar to the physiological properdin forms (in particular P2) than to Punfrac or Pn. The experiment was repeated 3 times with different time points, and the trend was consistent in all experiments. The results show that the functional ELISA is useful to detect the function of properdin in NHS.

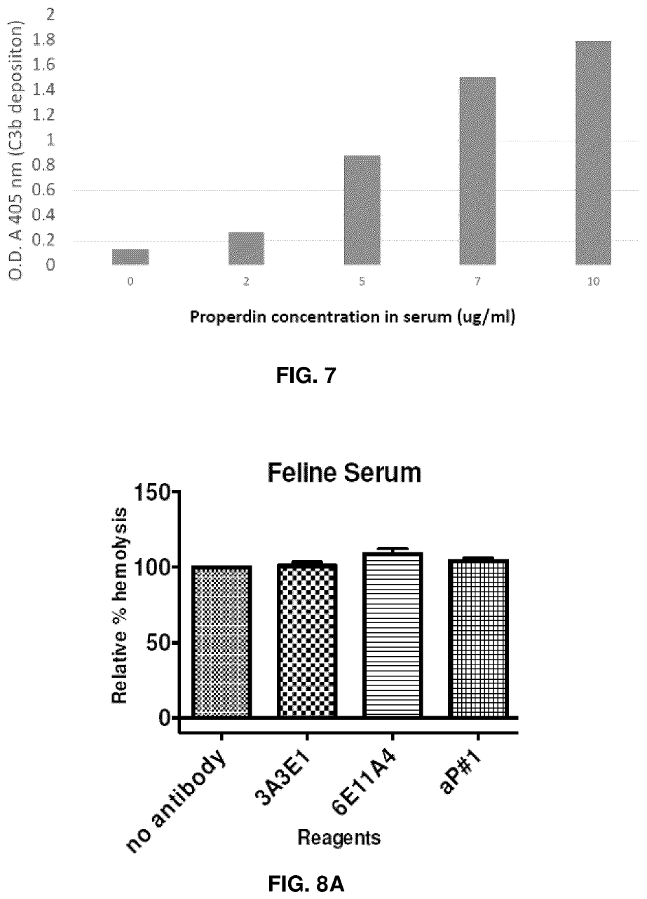

FIG. 7 shows that the functional properdin ELISA assay detects differences in activity between sera that have different properdin concentrations.

The method described herein provides a standardized assay which allows for the ability to measure function of properdin. It is now shown herein that this function is directly proportional to the concentration of properdin. Also shown herein are preferred times to measure function in human serum samples. The functional properdin ELISA assay is comparable to the in vitro red blood cell functional assay.

Kits

A kit for such functional properdin ELISA assay can include, for example:
1) a non-inhibitory antibody such as anti-properdin antibody (6E9E6), and
2) labeled anti-C3b antibody (commercial-source).

Instructions can also be included to note that properdin-depleted sera (requires −80° C. storage. Alternatively, instructions can be included for on-site reconstitution of lyophilized properdin-depleted sera.

The instructions can also note that properdin for control wells (requires −80° C. storage (or potential lyophilization).

Other optional components of the kit include: a buffer; detectable labels; etc. The various components of the kit may be present in separate containers or certain compatible components may be pre-combined into a single container, as desired.

In addition to above-mentioned components, a subject kit can include instructions for using the components of the kit to practice a subject method. The instructions for practicing a subject method are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g., compact disc-read only memory (CD-ROM), digital versatile disk (DVD), diskette, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate.

### Uses

The functional properdin ELISA assay is particularly useful for screening patient samples that have a low or no alternative pathway activity (AP50). As shown in FIG. 7, the result is proportional to the amount of properdin in the patient sera sample. Such assay simultaneously measures total AP50 activity just by adding the patient sample without EDTA, as well as can simultaneously measure the exact concentration of properdin in wells that receive another anti-properdin monoclonal antibody (such as 1G6D2) instead of the properdin-depleted sera, which would detect if the lack of function is due to lack of properdin in the sample.

The functional properdin ELISA assay is particularly useful for the detection of aggregates in purified properdin preparation (research) where it is important to distinguish between the aggregates and the physiological P forms since the commercially available properdin has both aggregates and the physiological P forms.

The final characterization step involved determining the species specificity of the monoclonal antibodies, using a modified alternative pathway hemolysis assay. The animal species whose properdin is recognized by the monoclonal antibodies can be used as experimental animal model for assessing properdin function in vivo.

Sheep erythrocytes (ES), which are normally protected by Factor H, were suspended in GVB= at approximately 1×10⁹ cells/ml. In each tube, ES (5×10⁶ cells/tube) in 5 mM MgEGTA or 10 mM EDTA, were incubated with PBS, recombinant competitive inhibitor of Factor H C-terminal domains 19-20 (rH19-20) (0-8.5 μM), and animal serum (i.e. rat, mouse, rabbit, guinea pig, goat, baboon, feline, ferret and canine) at a 40% concentration, in a total volume of 24 μl, for 20 minutes at 37° C. The tubes were then placed on ice and 200 μl cold GVBE was added to each tube to stop the reaction. The tubes were spun at 2000 g for 3 minutes at 4° C. The absorbance (Abs) of 200 μl of each supernatant was measured in a microtiter plate at 414 nm. The hemolytic activity was expressed as a % of hemolysis using the following equation: % of Hemolysis=[(Abs sample-Abs control)/(Abs maximum-Abs control)]×100. The “Abs sample” is the ES in 5 mM MgEGTA with or without rH19-20+PBS. The “Abs maximum” is the ES in 5 mM MgEGTA with H₂O+PBS (maximum lysis control). The “Abs control” is ES in 10 mM EDTA+ PBS+rH19-20. After graphing the results, the concentration of rH19-20 required to cause approximate 50% hemolysis was determined. An absence of lysis in the presence of the anti-properdin antibodies indicates that the properdin from the serum of the tested species is recognized by the anti-properdin antibody

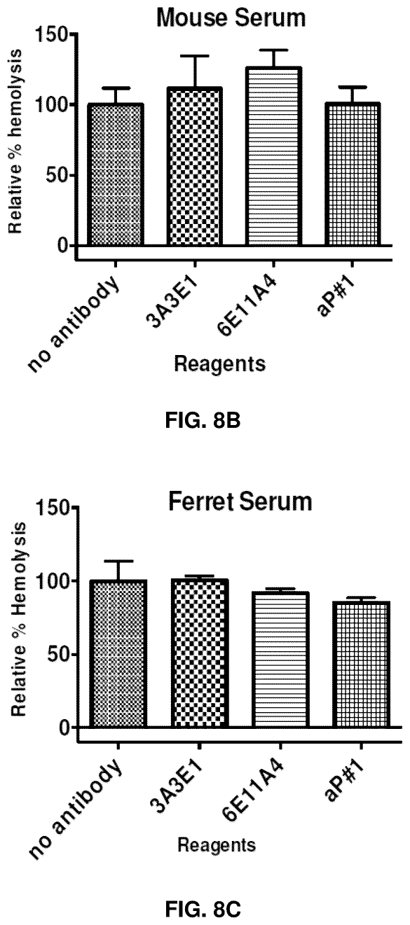

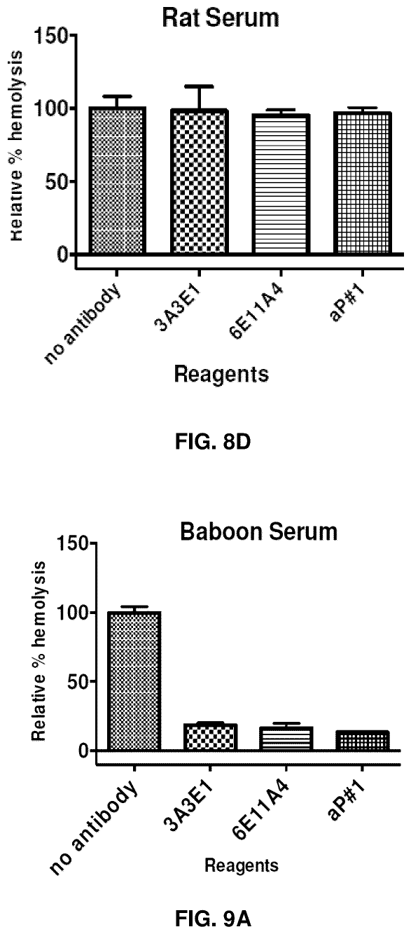

For example, properdin from feline serum (FIG. 8A), mouse serum (FIG. 8B), ferret serum (FIG. 8C) and rat serum (FIG. 8D) are not recognized by the monoclonal antibodies.

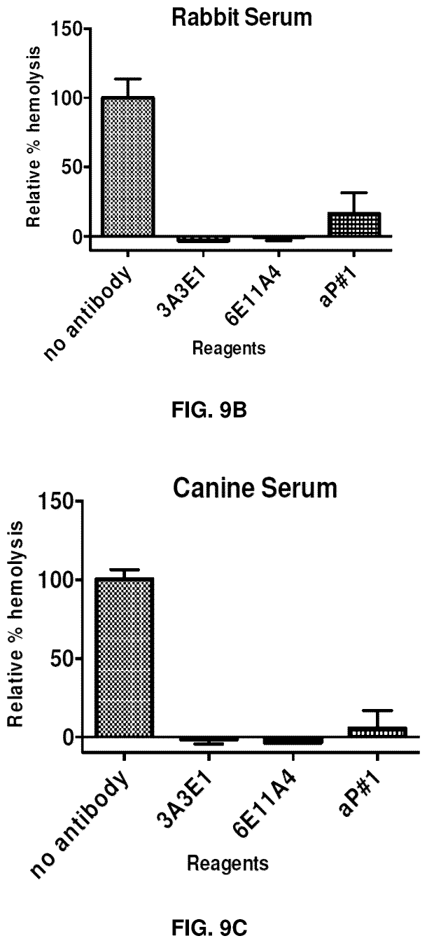

In contrast, properdin from baboon serum (FIG. 9A), rabbit serum (FIG. 9B) and canine serum (FIG. 9C) are recognized by the monoclonal antibodies.

### Example II

### Neutrophil-Derived Properdin has a Distinct Oligomeric Distribution

Properdin acts as an essential positive regulator of the alternative pathway of complement by stabilizing enzymatic convertases. Identical properdin monomers form head-to-tail associations of oligomers in a reported 20:54:26 ratio (most often described as an approximate 1:2:1 ratio) of tetramers (P4), trimers (P3), and dimers (P2), in blood, under normal physiological conditions. Oligomeric size is proportional to properdin function with tetramers being more active, followed by trimers and dimers. Neutrophils are the most abundant granulocyte, are recruited to inflammatory microenvironments, and are a significant source of properdin, yet the ratio of properdin oligomers released from neutrophils is unknown. The oligomer ratio of neutrophil-derived properdin could have functional consequences in local microenvironments where neutrophils are abundant and complement drives inflammation. In this example, the oligomer properties of neutrophil-derived properdin were investigated, as compared to that of normal human sera, using an ELISA-based method that detects function of properdin in a way that was proportional to the oligomeric size of properdin (i.e., the larger the oligomer, the higher the detected function). Unexpectedly, neutrophil-derived properdin had 5-fold lower function than donor-matched serum-derived properdin. The lower function was due to a lower percentage of tetramers/trimers and more dimers, indicating a significantly different P4:P3:P2 ratio in neutrophil-derived properdin (18:34:48) as compared to donor-matched serum (29:43:29). Release of lower-order oligomers by neutrophils may constitute a regulatory mechanism to control the rate of complement activation in cellular microenvironments. The factors that affect properdin oligomerization and how the predominant dimers in neutrophil-derived properdin assimilate to the ˜1:2:1 ratio found in serum are not currently known.

The complement system is an essential component of the immune system consisting of three pathways: classical, lectin, and alternative, which are each activated differently, and converge at the generation of C3b. The alternative pathway (AP) is a surveillance system that is always active, whereby C3 in blood spontaneously hydrolyzes to produce C3(H₂O) (C3 tickover mechanism). C3(H₂O) binds Factor B, which is then cleaved by mature Factor D to form a fluid-phase C3 convertase known as C3(H₂O)Bb that cleaves circulating C3 to C3b. C3b can bind covalently to hydroxyl or amino groups on nearby surfaces where it interacts with Factors B and D to form a surface-bound C3 convertase, C3bBb. C3 convertases cleave C3 to generate C3 fragments (C3a and C3b). It is also believed that the AP is primarily an amplification loop of C3b generated from the classical and lectin pathways with contributions from non-complement proteases. In order for the AP to carry out essential amplification of all three complement pathways, the C3 convertase of the AP is stabilized by a positive regulatory protein, properdin, increasing the half-life of the convertase by 5-10-fold. Subsequent binding of C3b near C3 convertases forms C5 convertases that cleave C5, leading to formation of C5a and the membrane-attack complex (C5b-9). The complement fragments that are generated during complement activation serve as potent proinflammatory mediators that are critical for opsonization, immune modulation, chemotaxis, and numerous other functions.

A fine balance between complement activation and regulation ensures the host is protected from danger such as infection, but not overwhelmed by excessive, harmful inflammation.

Regulation of the AP by negative regulators, both fluid-phase (AP: Factor H, Factor H-like protein 1; terminal pathway: clusterin, vitronectin) and membrane bound (AP: CRIg, CD35, CD46, CD55; terminal pathway: CD35, CD46, CD55, CD59), are essential for ensuring complement activity does not result in unintentional damage to the host. In contrast, in order to increase the effectiveness of the AP, properdin serves as an essential positive regulator by 1) stabilizing AP convertases, as mentioned above; 2) enhancing proconvertase (C3bB) formation; 3) competing with Factor I to prevent cleavage of C3b into the inactive form, iC3b; and 4) serving as a pattern recognition molecule to initiate AP convertase formation de novo by recruiting C3b or C3(H₂O), followed by Factors B and D; however, the relevance of this initiating phenomenon in vivo remains unknown. Properdin is an oligomeric glycoprotein, which forms dimers (P2), trimers (P3), and tetramers (P4) of head-to-tail, non-covalent associations of identical monomeric subunits. In healthy serum, these oligomers are present in a stable 20:54:26 ratio of P4, P3, and P2. Each properdin monomer contains 442 amino-acid residues arranged into six complete thrombospondin type 1 repeat (TSR1-6) domains and a Nterminal TGF-b binding (TB) domain. Properdin oligomers form ring-shaped vertices upon interaction of the TB domain and TSR1 from one monomer with TSR4, TSR5, and TSR6 from another monomer. These vertices interact with C3b and Bb of convertases (or Factor B of proconvertases) at a binding site formed by TSR5 and a large loop of TSR6. Properdin oligomerization directly correlates with properdin convertase-stabilizing function (i.e., P4 are more functional than P3, and P3 are more functional than P2). Electron microscopy indicates each properdin vertex concurrently binds a convertase, thus delineating the association between oligomer function and its stoichiometry. Simultaneous interactions of oligomeric properdin with multiple C3b molecules on an activator cell surface where C3b convertases are forming remain to be determined.

Properdin is present in serum at a concentration of 4-25 mg/ml. Unlike most complement proteins which are generated in the liver, properdin is released primarily from activated leukocytes. Cells known to either release or synthesize properdin mRNA include granulocytes, macrophages, monocytes, dendritic cells, primary T cells, mast cells, adipocytes, and endothelial cells. Neutrophils, the most abundant leukocyte, release properdin stored in secondary granules synthesized during bone marrow maturation in response to a variety of inflammatory agonists. Although it is accepted that properdin is present in serum in an approximate 1:2:1 ratio of P4:P3:P2, the molecular composition of properdin when released by leukocytes is unknown. At local sites of inflammation, where neutrophils are abundant and subject to cytokine stimulation, the distribution of oligomers of neutrophil-derived properdin may further modulate local complement activity, beyond the increased properdin concentration.

In this example, the ability of properdin to capture and generate convertases de novo was utilized to develop an ELISA-based technique to evaluate the function of properdin in biological samples with a readout that correlated with the oligomeric distribution of properdin in the sample. For the first time, the oligomeric distribution of properdin released from neutrophils was characterized using this assay and confirmed by size exclusion chromatography. The assay revealed that the function of neutrophil-derived properdin was significantly lower than donor-matched serum properdin and the decreased function was confirmed to be due to the presence of predominantly P2 and P3.

### Materials and Methods

### Buffers

The following buffers were used: Gelatin veronal buffer (GVB=; 5 mM veronal, 145 mM NaCl, 0.004% NaN₃, 0.1% gelatin, pH 7.3), MgEGTA [0.1 M MgCl₂ and 0.1 M EGTA (ethylene glycol tetraacetic acid), pH 7.4], 0.5 M EDTA (0.25 M EDTA [ethylenediaminetetraacetic acid] disodium salt-2H₂O and 0.25 M EDTA tetrasodium salt-2H₂O, pH 7.4), GVBE (GVB=, 10 mM EDTA), phosphate buffered saline (PBS; 10 mM NaH₂PO₄, 145 mM NaCl, pH 7.4), Hanks' balanced salt solution with Ca²⁺ and Mg²⁺(HBSS++; Gibco), and ELISA dilution buffer (PBS+0.05% tween+1% bovine serum albumin (BSA)).

### Serum and Complement Proteins

Normal human serum (NHS) was purchased from Innovative Research or produced using healthy donors. The Institutional Review Board from the University of Toledo College of Medicine and Life Sciences approved the protocols, and written informed consent was obtained from all donors, in accordance with the Declaration of Helsinki. Properdin-depleted and C3-depleted serum were purchased from Complement Technology Inc. Unfractionated pure properdin was purified from normal human plasma as described previously. Rabbit erythrocytes (ER) were prepared from blood obtained from Rockland Immunochemicals.

### Antibodies

The following antibodies were used: mouse monoclonal immunoglobulin G (IgG)1 anti-properdin 3A3E1 (biotinlabeled) and 6E9E6 (unlabeled) antibodies developed as previously described and biotinylated mouse IgG1 anti-human C3/C3b/iC3b monoclonal (clone 7C12) (Cedarlane).

### Human Neutrophil Cell Isolation and Isolation

Human whole blood was collected via venipuncture from healthy donors. The Institutional Review Board from the University of Toledo College of Medicine and Life Sciences approved the protocols, and written informed consent was obtained from all donors, in accordance with the Declaration of Helsinki. Blood was drawn into BD vacutainer tubes containing 12 mg K3 (tripotassium) EDTA and polymorphonuclear (PMN) cells were isolated by using a Polymorphprep™ gradient solution (Axis Shield) following the manufacturer's instructions. PMN cells (5.0×10⁷ cells/ml) in 0.2% BSA/HBSS++ buffer were activated using 20 ng/ml of phorbol 12-myristate 13-acetate (PMA) (Enzo Life Science) for 30 min at 37° C. The PMNs were centrifuged at 600×g for 10 min. The supernatant was removed and centrifuged at 13,000 g for 2 min to remove cell debris. PMNs without PMA treatment were prepared as a control. The supernatants from PMA-activated and non-activated PMNs were assayed for properdin concentration, as indicated below.

### Sandwich ELISA to Quantify Properdin Concentration

Properdin concentration of test samples was assayed by a sandwich ELISA as previously reported. The samples (i.e., fractions collected from: pure properdin, NHS, neutrophil supernatant, and donor-matched serum) were tested at varying dilutions in ELISA dilution buffer, as indicated in the figure legends. Other samples tested, but not shown, include neutrophil supernatant samples (diluted 1/80, 1/160, and 1/320), donor-matched serum, NHS, and C3-depleted serum (diluted 1/800, 1/1600, 1/3200, and 1/6400).

Separation of Properdin Oligomers from Native Properdin and Biological Samples

Physiological oligomeric forms of properdin P2, P3, P4, and non-physiological aggregated properdin (Pn) were isolated from purified properdin by size exclusion chromatography as previously reported with minor modifications. Briefly, 200 mg purified properdin in a total volume of 400 ml in PBS was loaded onto a Phenomenex BioSep 5 mM SEC s4000 500 Å liquid chromatography column (600×7.8 mm) with guard column (75×7.8 mm) and was eluted at a flow rate of 0.3 ml/min in PBS. Fractions were collected in 250 ml volumes in glass tubes (12×75 mm) that were previously blocked with 500 ml GVB= overnight at 4° C. and then emptied prior to sample collection. Collected fractions were stored at 4° C. and used within one week of separation. Similarly, physiological oligomeric forms of properdin were separated from NHS and neutrophil supernatant by size exclusion chromatography by diluting the sample to contain 4 mg or 150 ng properdin (properdin concentration determined by sandwich ELISA) with 1% BSA and 20 mM EDTA in PBS, and syringe-filtered using 0.20 mM regenerated cellulose filter membranes (Phenomenex). Samples (400 ml final volume) were loaded onto a size chromatography column as described for purified properdin. For all samples that were run through the size exclusion column, the concentration of properdin within each fraction was determined using the sandwich ELISA as described below. A visual representation of the ratio of properdin oligomers in the sample was constructed by graphing the properdin concentration of each fraction. Peak fractions and no more than 3 fractions to the left or right of the peak fraction were selected for further analyses (i.e., properdin functional ELISA, hemolytic assay) of purified oligomers.

### Quantification of Properdin Oligomeric Ratios in Biological Specimens

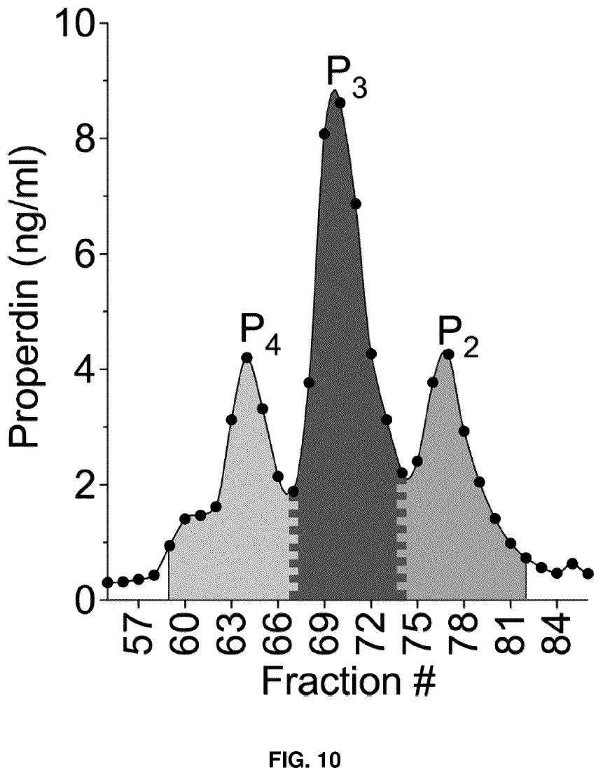

A systematic approach for determining the fraction boundaries of the properdin oligomer forms within each sample was applied using the plotted concentration data (method described above) for each fraction. FIG. 10 shows properdin concentration in each fraction derived from size exclusion chromatography of NHS (from donor #4). The width of the P4 peak was considered five fractions to the left of the P4 top fraction and all fractions to the right of the top fraction up until the fraction present in the valley between the P4 and P3. The fraction present at the valley between peaks was assigned to both P4 and P3 by dividing the concentration of that fraction equally between the two properdin oligomers. The width of the P2 peak was considered five fractions to the right of the P2 top fraction and all fractions to the left up until the valley between the P2 and P3 peaks. The fraction present at the valley between these peaks was assigned to both P2 and P3 by dividing the concentration of that fraction equally between the two properdin oligomers. The width of the P3 peak was defined as all fractions in between the P4/P3 valley and the P3/P2 valley. After assigning fractions to properdin oligomers, the concentration from fractions belonging to each properdin oligomer form was totaled. Then, all fractions from all oligomers were summed for a grand total. The ratio of properdin oligomers was determined by dividing the total concentration of each properdin oligomer by the grand total and multiplying by 100. Comparable results were obtained when fitting the concentration data described in FIG. 10 to three peaks using computer-generated curves applying Fityk software assuming Gaussian distributions for each species.

### Development of an ELISA-Based Assay to Assess Properdin Function

The following assay parameters were varied and overall optimal conditions allowing the greatest signal to noise ratio were chosen with respect to the concentration of sensitizing anti-properdin monoclonal antibody 6E9E6, properdin concentration in the sample, concentration of properdin-depleted sera, properdin function reaction time, concentration of anti-C3b monoclonal antibody, dilution of horseradish peroxidase-streptavidin, and absorbance read time. Two versions of the assay that result in equivalent outcomes (using either medium-binding full-area or high-binding half-area ELISA plates) were standardized and carried out as described below.

### Properdin Functional ELISA to Assess Properdin Function in Purified Properdin and in Biological Samples

A 96-well, high-binding, half-area, ELISA plate (Greiner Bio One) was coated with 50 ml/well of 1 mg/ml non-inhibitory anti-properdin monoclonal antibody 6E9E6 diluted in PBS overnight at 4° C. The plate was washed 4× with 130 ml/well 1× PBS containing 0.05% Tween-20 (PBST) and blocked for 2 h at 37° C. with 125 ml/well 3% BSA in PBS. Properdin sources were diluted to equivalent concentrations within each assay (ng/ml; indicated in figures) in ELISA dilution buffer and 20 mM EDTA then added to the plate and incubated at 37° C. for 1 h for properdin capture by the coating antibody, 6E9E6. Background was determined by adding properdin-depleted serum (for serum), PBS (for pure properdin), or 0.2% BSA with HBSS++(for neutrophil supernatant), instead of a properdin source. The plate was washed 4× with 130 ml/well PBST and while on ice, 50 ml/well properdin-depleted serum diluted 1/10 in GVB= with 5 mM MgEGTA was added to the plate while cold water was added to empty wells not containing any sample to ensure even temperature distribution across the plate. The plate was incubated for 45 min at 37° C. 12.5 ml/well cold 0.1 M EDTA was added to all wells to stop complement activity, and the plate was washed 4× with 130 ml/well PBST. C3b deposited covalently on the plate was detected with 50 ml/well biotinylated anti-C3b monoclonal antibody at 200 ng/ml for 1 h at 37° C. The plate was washed 4× with PBST and incubated for 45 min at 37° C. with horseradish peroxidase-streptavidin (Cedarlane) diluted 1:2500 in ELISA dilution buffer. The plate was washed 4× with 130 ml/well PBST and incubated for 30 min with 10-parts ABTS to 1-part ABTS enhancer at room temperature and absorbance was read at 405 nM on a Tecan Infinite M200 spectrophotometer. During each incubation, the plate was sealed with a plastic film. Data was normalized by dividing C3b deposition of each sample by C3b deposition of an internal plate control, NHS.

### Properdin Functional ELISA to Assess Properdin Function in C3-Depleted Serum, and Neutrophil Supernatant and Serum Matched Samples

This assay used full-area, medium-binding plates (Greiner Bio One), resulting in differences in some of the ELISA parameters as compared to section 2.8.1 as follows: (a) all volumes at all steps were doubled; (b) the plates were coated with 10 μg/ml of the 6E9E6 antibody; (c) the following sources of properdin were used: C3-depleted serum, NHS, neutrophil supernatant, or donor-matched serum; (d) properdin-depleted serum was used at 1/10-1/16 dilution and the incubation that followed was 90 min; (e) the biotinylated anti-C3b antibody was used at 20 ng/ml; and (f) the horseradish peroxidase-streptavidin was diluted 1/5000.

### Properdin Functional Hemolytic Assay to Assess Properdin Function in Purified Properdin Oligomers and in Biological Samples

2×10⁷ ER were mixed with GVB= and the following reagents at the indicated final concentrations: 2.5 mM MgEGTA or 10 mMEDTA, 0-60 ng/ml of properdin from NHS, pure properdin, or properdin oligomers from pure properdin (P2, P3, and P4 and Pn), and 20% properdin depleted serum in a total 50 ml volume. Next, the mix was incubated for 10 min at 37° C., mixing the tubes every 5 min. The tubes were then placed on ice and 200 ml of cold GVBE was added to each tube to stop the reaction. The tubes were spun at 2000 g for 3 min at 4° C. The absorbance of 200 ml of each supernatant was measured in a microtiter plate at 414 nM. The % of hemolysis was calculated using the formula: [(A414-background A414 in the presence of EDTA)/(maximum A414 determined by water lysis-background A414 in the presence of EDTA)]×100.

### Statistical Analyses

Data was analyzed by unpaired t-test, one-way ANOVA with Tukey's post-test, or two-way ANOVA with Bonferroni's post-test using GraphPad Prism version 9.2.0 for Mac OS X (GraphPad Software, San Diego, California, USA), or by ANCOVA analysis followed by Tukey's post hoc test using SAS 9.4 (SAS Institute Inc., Cary, NC, USA) (as indicated in figure legends or results section). P values less than 0.05 were considered significant.

### Development of an ELISA-Based Method to Characterize Properdin Function in Biological Samples

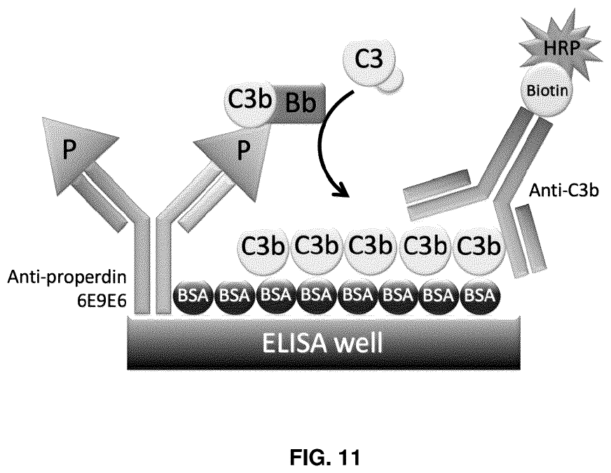

Given that properdin oligomer distribution influences overall properdin function (P4 have greater function than P3, and P3 have greater function than P2), an assay that could detect functional differences in biological samples was developed. This was accomplished using a multiplexed (96-well format), ELISA-based approach (FIG. 11). In order to measure the function of properdin captured from a biological source, a non-inhibitory, anti-properdin monoclonal antibody (6E9E6) was used as a capture antibody. In addition, to ensure that equivalent amounts of properdin (ng/ml) could be captured, irrespective of the oligomerization state, 6E9E6 was used because it did not detect differences between P2, P3, and P4. Before the next step, the concentration of properdin in the samples was determined using the sandwich ELISA described in the materials and methods section. The next step of the functional properdin ELISA was adding to the 6E9E6-sensitized wells equivalent concentrations of properdin within biological samples (i.e., serum, neutrophil supernatant) or pure properdin, in the presence of EDTA (to prevent complement activation). After properdin capture, the plate was washed, so that only captured properdin remained, and incubated with properdin-depleted serum with MgEGTA (to facilitate AP activity only). Using properdin-depleted serum as a source of complement proteins for de novo convertase formation on the captured properdin, ensured properdin activity was only contributed by the captured properdin. The efficiency of properdin to initiate AP activity (i.e., properdin function) was determined by the level of C3b covalently bound to the BSA coating the plate, which was detected by an anti-C3b monoclonal antibody. The inter- and intra-assay coefficient of variation (CV) for the assay format described in the materials and methods section, was 19.6% and 5.3%, respectively. For the format, the interassay and intra-assay CV was 9.7% and 6.5%, respectively. Additional validation is described below.

### The Properdin Functional ELISA can Detect Differences Between Properdin Oligomers

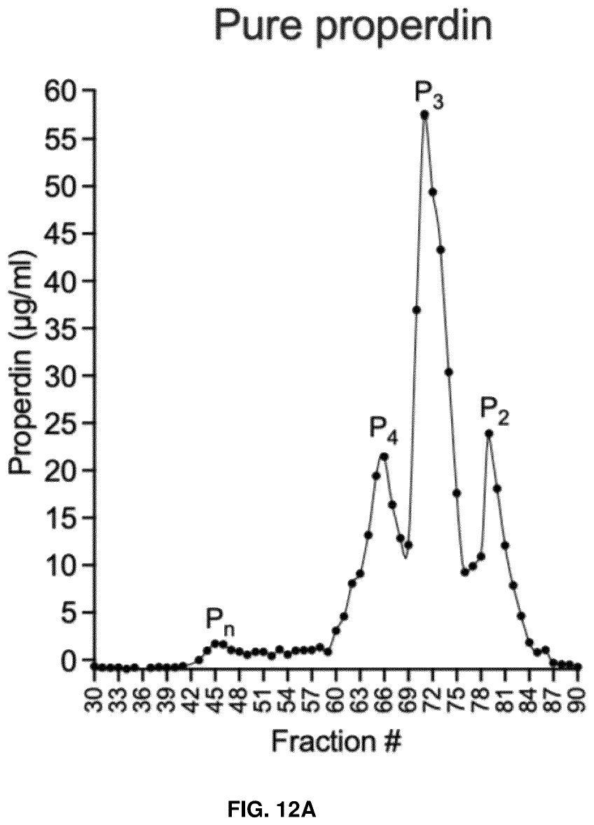

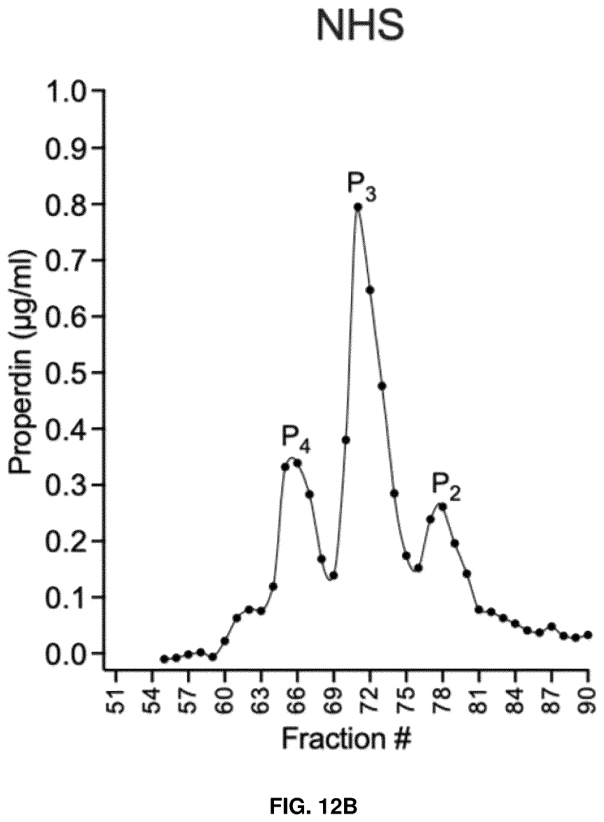

The functional ELISA was validated by evaluating the ability of different captured properdin oligomers to promote C3b deposition. For this purpose, size exclusion chromatography was used to separate physiological oligomeric forms (P2, P3, and P4) and non-physiological aggregates (Pn) from unfractionated pure properdin. Physiological oligomers in the context of NHS were also separated. After fractionating the samples containing properdin, the concentration of properdin in the collected fractions was determined by sandwich ELISA and the resulting concentrations were plotted to visualize the oligomer distribution in the sample for purified properdin (FIG. 12A) and NHS (FIG. 12B). Both properdin sources showed P4, P3, and P2 peaks at similar ratios (i.e., ˜18:57:25 for unfractionated pure properdin and ˜20:52:27 for NHS) and the unfractionated pure properdin contained a small Pn peak.

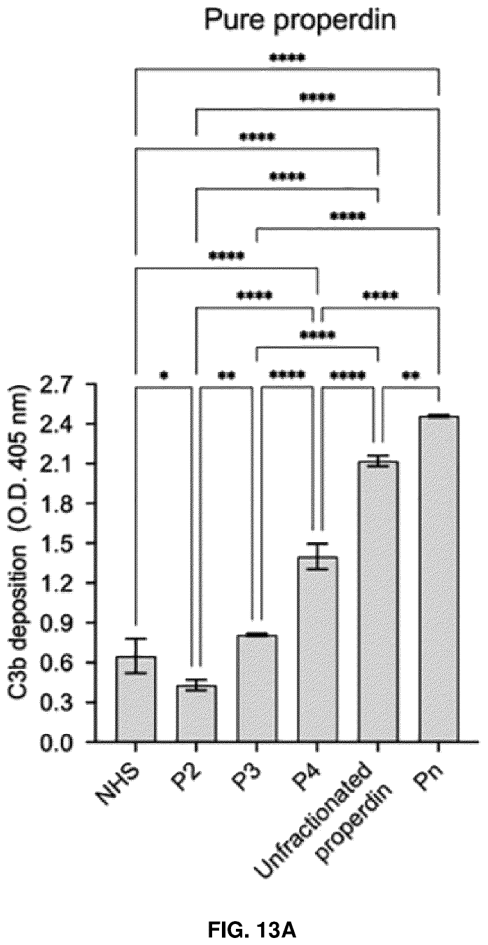

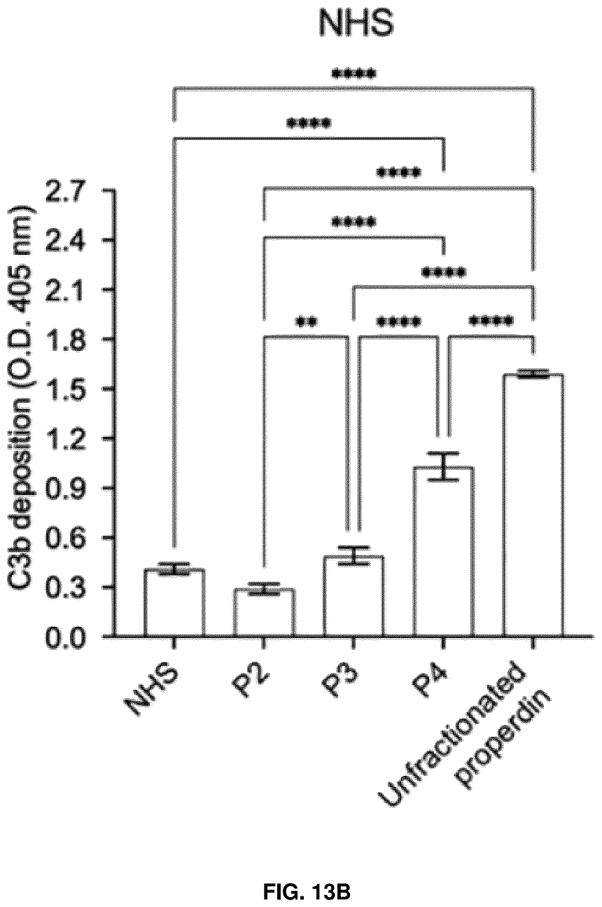

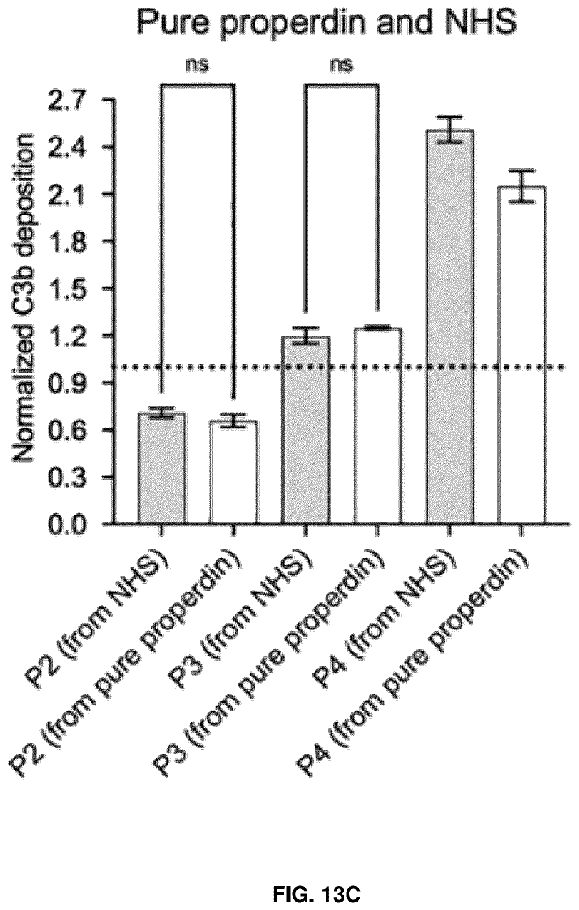

The function in the selected chromatography fractions of each separated properdin oligomers was assayed at equivalent concentrations in the functional ELISA as described in the materials and methods section. The assay detected significant differences in function between individual properdin oligomers separated from both pure properdin and serum. P2 from purified properdin were 30% and 53% as active as P4 and P3 from purified properdin, respectively. In the case of oligomers separated from serum, P2 were 37% and 64% as active as P4 and P3, respectively. P3 from pure properdin and serum were 58% as active as P4 from pure properdin and serum. The function of properdin in NHS was closest to pure P3 and physiological P2/P3, since NHS lacks the Pn forms. Pn, which are the highly aggregated, non-physiological oligomers of properdin, showed the highest function, and unfractionated pure properdin, which contains Pn and the physiological oligomers, resulted in the second highest function (FIGS. 13A-13B).

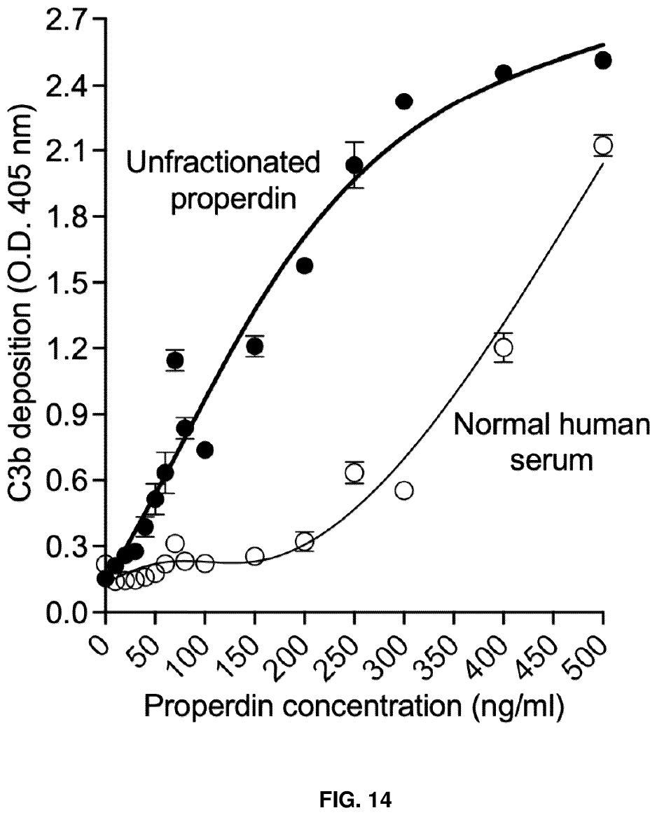

The utility of the functional ELISA to detect dose-dependent functional differences between unfractionated pure properdin that has P2, P3, P4, and aggregated Pn forms, and serum-derived properdin that lacks Pn forms, was also tested. The function of 16 concentrations (0-500 ng/ml) of unfractionated pure properdin and properdin within serum was assessed. This resulted in a concentration-dependent increase in both serum-derived and unfractionated pure properdin with properdin in serum having lower function at the tested doses (FIG. 14), indicating that the properdin functional assay detected dose-dependent increases in properdin function and wide functional differences between samples with distinct oligomeric composition.

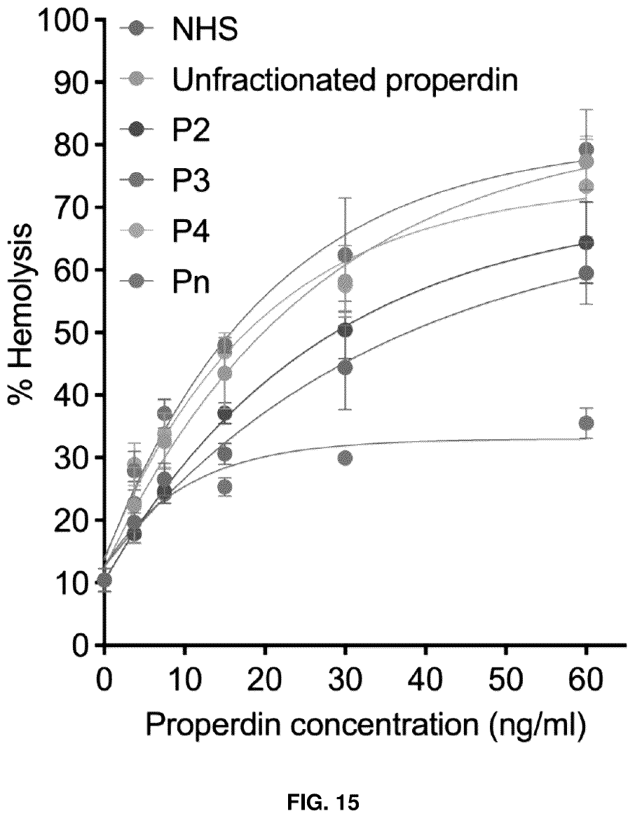

The hemolytic properdin functional assay does not detect greater properdin function when higher order properdin oligomers are present. To determine if another assay, in addition to the functional ELISA, can detect functional differences in samples with distinct oligomer composition, the function of unfractionated pure properdin, properdin in NHS, and properdin in individual properdin oligomers (P2, P3, P4, and Pn) was also evaluated in a properdin functional hemolytic assay using ER, which are susceptible to AP lysis (FIG. 15). Using ANCOVA analysis with Tukey's post hoc test, the data indicate that although NHS has similar function as P2 at all concentrations tested, no differences were observed between unfractionated pure properdin (that contains contaminating Pn) and P3, and P4 had the same activity as P3. In addition, Pn resulted in the lowest hemolysis, most likely due to consumption of complement in the serum, as described previously for high order properdin oligomers. Overall, the data indicates that under the conditions tested, the hemolytic assay cannot detect oligomer-dependent differences in properdin function.

Properdin Functional ELISA does not Capture Serum-Derived Properdin in Combination with C3(H₂O) or C3b

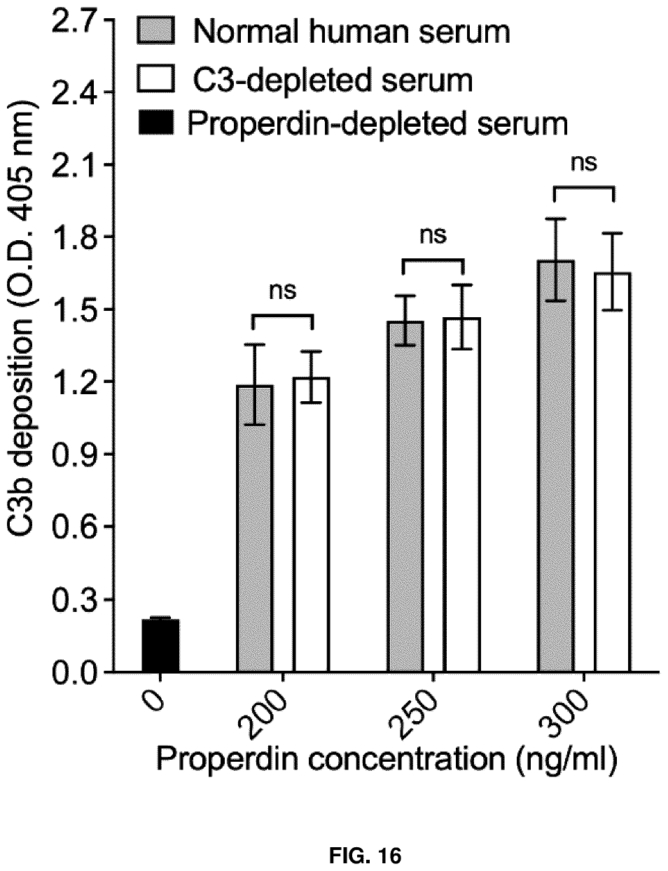

The readout of the functional ELISA is the deposition of C3b generated by C3 convertases initiated by properdin. It is known that properdin can be found complexed to certain proteins in serum, such as C3(H₂O), C3 fragments, clusterin, IgG, and C3b-C3b-IgG complexes. In order to rule out interference from C3b deposition being potentially contributed by C3(H₂O) or C3b associated with properdin during the step when properdin was captured from serum, the function of properdin within NHS was compared to properdin captured from C3-depleted serum using the functional ELISA as described in the materials and methods section. The assay detected no differences in function between serum-derived properdin and properdin derived from C3-depleted serum at the tested concentrations of properdin (FIG. 16), indicating that if any C3(H₂O) or C3b was being captured with the properdin it does not interfere in the assay itself.

### Assessment of Neutrophil-Derived Properdin Oligomeric Distribution

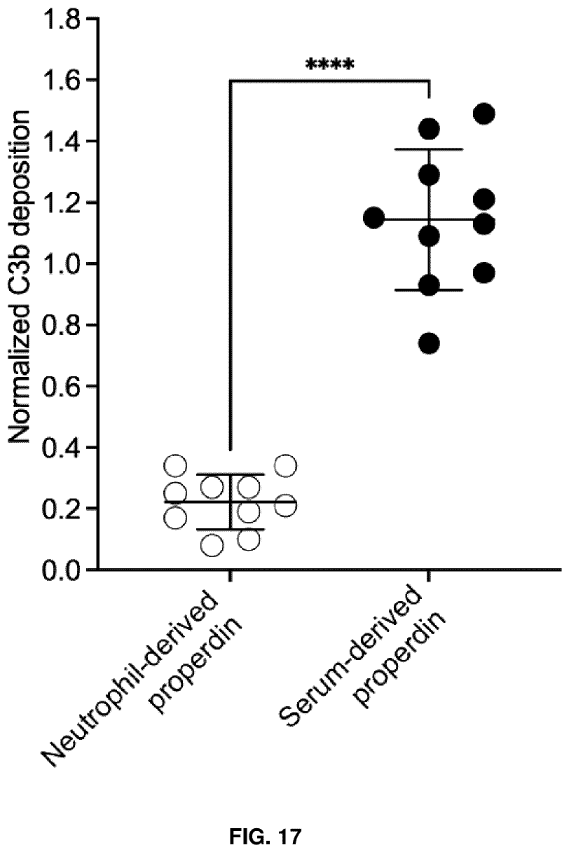

Human neutrophil-derived properdin has lower function than properdin in donor-matched serum as detected by the properdin functional ELISA. The ability of the functional ELISA to detect functional differences between samples that contain different oligomer composition (e.g., between NHS and pure properdin or individual oligomers), indicates that the assay can serve as a first indication of differences in distribution of properdin oligomers within a sample. Thus, the assay was used to screen the function of properdin within activated neutrophil supernatants isolated from the blood of heathy human volunteers. Neutrophils release properdin upon activation and are an important source of serum properdin. Activated neutrophils release properdin stored within secondary granules into the local, cellular microenvironments, which may contribute to the overall inflammatory response within these environments. However, the distribution of neutrophil-derived properdin oligomers (which would correlate to properdin function) is unknown. To address this gap, the function between serum-derived and neutrophil-derived properdin was assessed. To carry out a comparison at the individual level, serum and neutrophils from the same donor were collected. The neutrophils were activated using PMA, the cellular supernatants were collected, and the concentration of properdin in the supernatants was determined by using the sandwich ELISA as described in the materials and methods section. The concentration of properdin in serum was between 8-19 mg/ml and 649-923 ng/ml in activated neutrophil supernatant. On average, the concentration of properdin released from the cellular supernatant of non-activated neutrophils was 170+/−128 ng/ml. The function of properdin in serum and in the neutrophil supernatants was next assessed at equivalent properdin concentrations in the functional ELISA as described in the materials and methods section. The function for neutrophil-derived properdin was significantly lower (an average 5-fold lower) than serum-derived properdin (FIG. 17). Given that properdin function quantified in the functional ELISA was a property of oligomerization (FIG. 13), whether the lower function associated with neutrophil-derived properdin was due to an alteration in the ratio of properdin oligomers distinct from the approximate 1:2:1 ratio reported for serum-derived properdin was assessed.

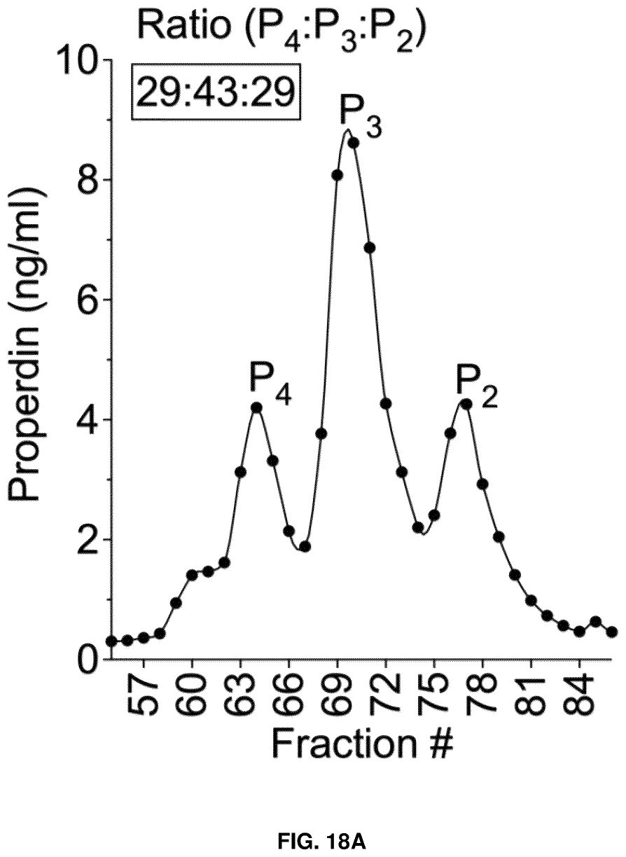

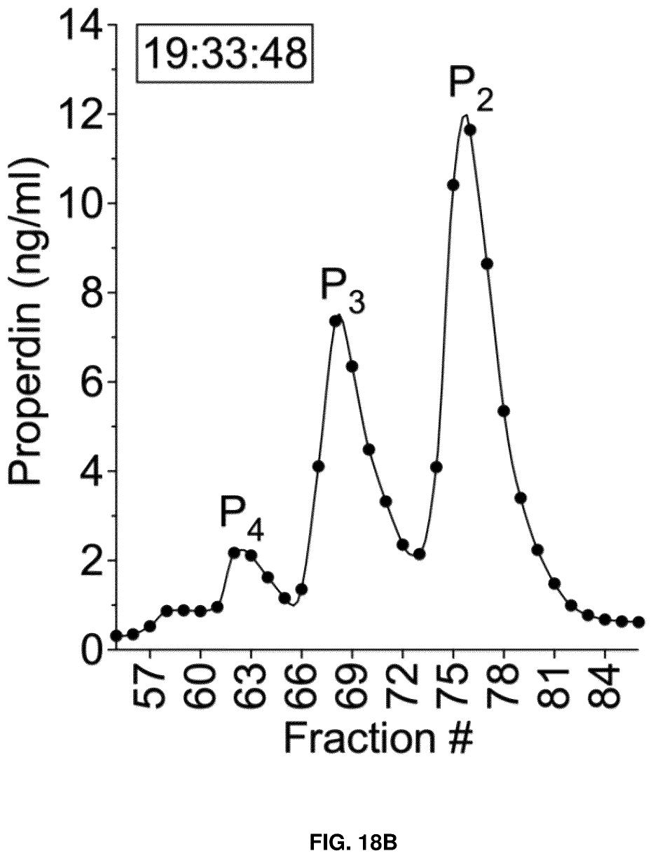

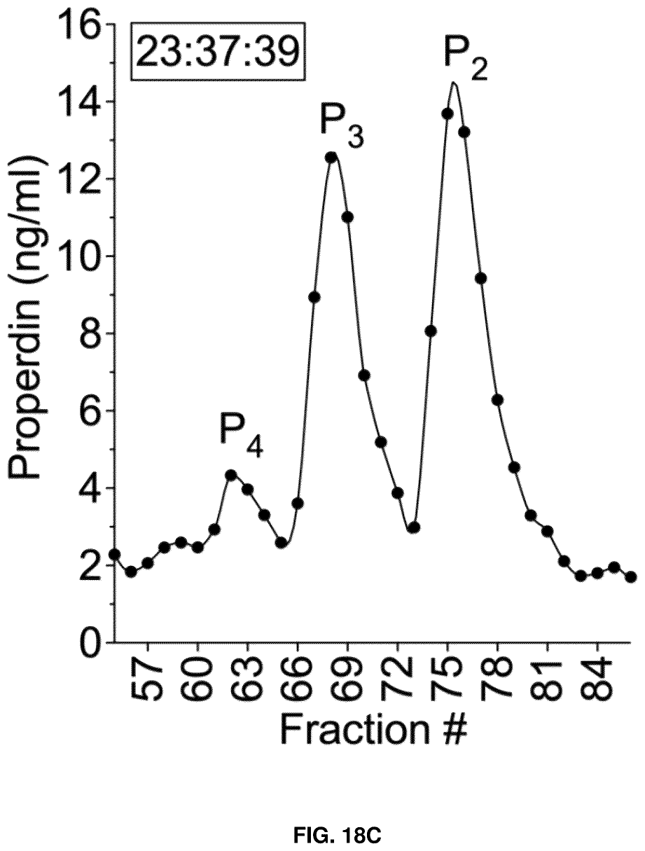

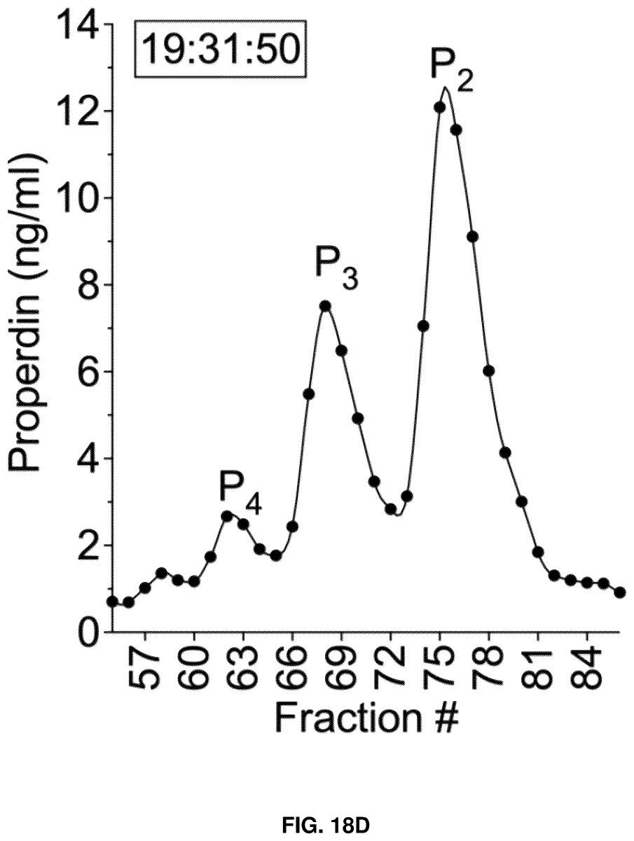

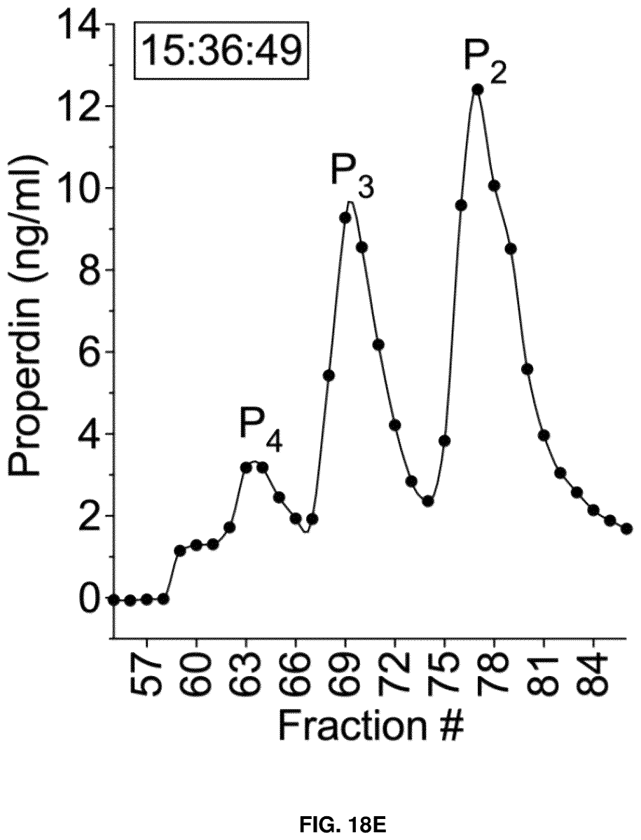

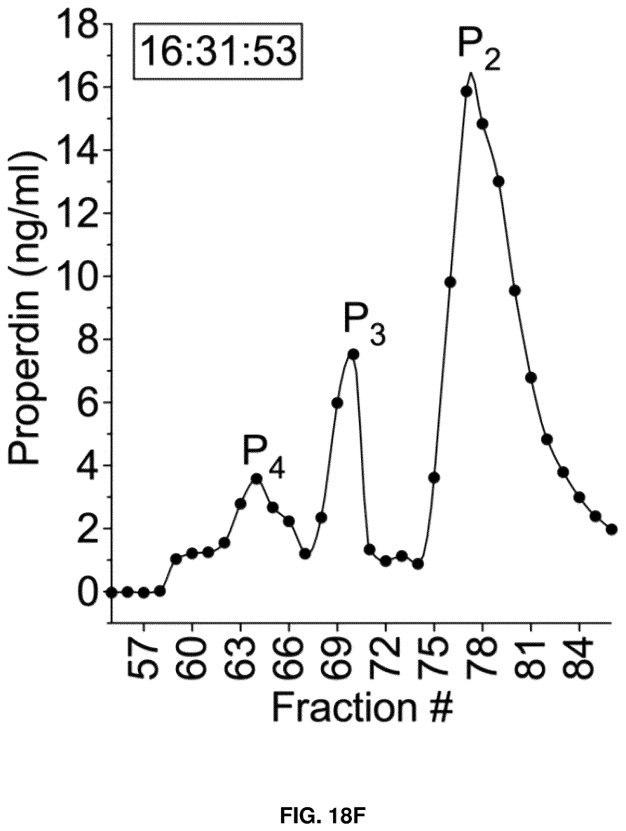

The function of neutrophil-derived properdin was due to a properdin oligomer ratio distribution that was different from serum, skewed towards dimers. To address whether the lower function of neutrophil-derived properdin, as compared to serum, was due to differences in oligomer distribution, size exclusion chromatography coupled with quantification of properdin of the fractions were performed and oligomeric ratios for these samples were calculated as described in the materials and methods section. The ratio of properdin oligomers derived from serum (FIG. 18A) was a ratio of 29:43:29 (P4:P3:P2). The ratio of properdin oligomers in activated neutrophil supernatants from 5 donors (FIGS. 18B-18F) was characterized by a lower amount of P4/P3 and higher amount of P2 resulting in oligomeric ratios of 19:33:48, 23:37:39, 19:31:50, 15:36:49, and 16:31:53 (average 18:34:48). Overall, the data indicate that the ratio of properdin oligomers released from PMA-activated neutrophils was different from NHS and favors lower-order forms.

### DISCUSSION

Neutrophils account for a significant source (˜19-32%) of circulating properdin. It is likely that neutrophils are major contributors of properdin in inflammatory microenvironments because they are the first cells recruited to sites of infection where they facilitate the clearance of microorganisms via phagocytosis, the release of cytotoxic molecules from secondary granules, and the formation of neutrophil extracellular traps (NETs). Neutrophils also participate in adaptive immune regulation through interactions with antigen-presenting cells and lymphocytes. Neutrophils secrete properdin stored within granules in response to a variety of inflammatory agonists. Properdin carries out a series of essential canonical and noncanonical functions including promotion of: oxidative burst in neutrophils, complement activation on NETs, and complement-mediated platelet-granulocyte formation, and also can serve as a ligand for natural killer cells, leading to antimicrobial outcomes independent of complement activation. Properdin released from neutrophils can bind activated platelets and apoptotic T-cells. Any of these functions may affect inflammatory states both in circulation and especially in cellular microenvironments. Because the magnitude of the function of properdin was directly related to the ratio of properdin oligomers (FIG. 13), identifying the oligomerization state of properdin when it is released from neutrophils is important, yet remained unknown. In this example, an ELISA-based assay that can measure the function of properdin was utilized. This ELISA-based assay was dependent on the oligomerization state of properdin in the sample, which allowed this question to be addressed.

Properdin is known to be essential for stabilizing AP convertases; however, properdin has been shown, via surface plasmon resonance, to also serve as a focal point for de novo convertase assembly in vitro. This convertase-initiating property was used to develop the properdin functional assay. The assay utilized a non-inhibitory, monoclonal, anti-human properdin antibody (6E9E6) to capture properdin from various sources including pure properdin, serum, and neutrophil supernatant (FIG. 11). 6E9E6 was specifically chosen given its non-inhibitory nature and the fact that it cannot distinguish between properdin oligomers when assayed at equivalent ng/ml concentrations. Thus, this allows the readout of the assay (C3b deposition) to represent properdin function alone, as a property of oligomer sizes present in the sample.

The function of properdin oligomers derived from either pure properdin or from serum was evaluated to validate the functional assay. For this purpose, properdin from both sources was fractionated into oligomeric forms by size exclusion chromatography, followed by quantification of properdin in each fraction (FIG. 12). The ratio of properdin oligomers in both sources followed the approximate 1:2:1 ratio of P4, P3, and P2 and pure properdin contained a small peak consisting of non-physiological aggregates (Pn) that form after repeat freeze/thaw cycles or prolonged storage whereas NHS contained a minor peak prior to P4, which may indicate the presence of pentamers or hexamers, which have been identified in NHS by others. The addition of the sandwich ELISA for determining concentration of the fractions, following properdin oligomeric separation within serum by size exclusion chromatography, became necessary to define the oligomers within the complex mixture of proteins found within serum. The functional properdin ELISA and size exclusion chromatography are complementary techniques. The ELISA served as an initial high-throughput assay that allows testing of several samples (>28, in triplicate, plus appropriate controls) for detection of differences in overall oligomer-related function between samples, while the size exclusion chromatography was run on individual selected samples to confirm oligomer distribution in relation to the ELISA-based function. In addition, the functional properdin ELISA, given its ability to detect function proportional to size while using very small amounts of properdin (ng), may serve as a practical screening tool for assuring absence of contaminating Pn in purified properdin preparations.

When assessing the function of properdin oligomers in the functional ELISA, there was a significant increase in C3b deposition between each properdin oligomer (P2, P3, and P4) collected from both purified properdin and serum (FIGS. 13A-13C). In both sources of properdin, P2 was less active than P3, and P3 was less active than P4. The function of serum-derived properdin was comparable to the function of P3 from purified properdin and P2/P3 in the context of serum, as serum-derived properdin contains predominantly P3, and does not contain non-physiological Pn. Properdin function was the highest in non-physiological, aggregated properdin (Pn), followed by unfractionated pure properdin that contains a mixture of physiological and non-physiological oligomers. Although the Pn fraction only represents a small amount of the total properdin within unfractionated properdin, there are several characteristics of properdin that indicate Pn is a highly potent complement activator: (a) Pn elutes as a minor peak (FIG. 12A) with an approximate molecular mass greater than 106 Da; (b) structural studies indicate convertases are formed at vertices of properdin oligomers; (c) the Pn fraction, when added to serum, completely consumes complement. Altogether, this indicates that Pn acts similar to an activating surface, likely due to the high, variable number of available vertices and thus, even small amounts of Pn in the context of unfractionated properdin may have exponential effects on the resulting C3b deposition.

The assay was further validated by demonstrating that it can detect concentration-dependent functional differences between unfractionated pure properdin and serum-derived properdin (FIG. 14). Unfractionated pure properdin, which contains highly aggregated oligomers, had higher function than properdin derived from serum across different properdin concentrations (50-500 ng/ml). It was evident that at the lower and higher concentrations, the margin of difference between these two properdin sources narrows. Hence, for evaluating properdin function, the concentration at which properdin is prepared during the capture step must be considered. Altogether, the data indicate the convertase-initiating/stabilizing capacity of properdin oligomers increased with size and highlights the feasibility of applying the functional ELISA to detect functional differences between properdin sources and, in turn, determine whether the ratio of properdin oligomers is skewed towards P2 or P4.

It has been shown previously that binding of purified properdin to necrotic cells and zymosan is dose-dependently inhibited when serum is added to the pure properdin. Likewise, when necrotic cells and zymosan are incubated with NHS (heat inactivated or in presence of EDTA, to eliminate complement activity), native properdin in the serum does not bind. The functional assay described herein does not rely on the ability of properdin to bind to a surface. Instead, it utilizes a noninhibitory monoclonal anti-properdin antibody to capture properdin from any sample, followed by washing, which leaves only properdin that has been captured. Thus, the captured properdin provides a site for de novo convertase formation when properdin-depleted sera is added in the following step. It remains possible that hypothetical inhibitory factors could be pulled down with the properdin during the capture step. However, FIG. 13C shows no difference in the level of C3b deposition between properdin oligomers (P2 and P3) separated from pure properdin or captured from NHS, indicating the absence of inhibitory factors in serum that would have contributed to differences in properdin function between these two sources. Properdin function is higher in P4 captured from NHS when compared to pure properdin (FIG. 13C), which while unexpected, rules out the presence of inhibitory factors in serum that would contribute to reduced properdin function. To rule out the possibility that properdin was being captured along with C3(H₂O) or C3 fragments in biological samples such as serum, which could interfere in the outcome, the functional properdin ELISA was carried out with C3-depleted serum as a source of properdin and the results were compared to NHS as a properdin source, at equivalent concentrations (FIG. 16). The deposition of C3b was not significantly different between properdin sources, indicating that all the observed function was due to de novo convertases forming on the captured properdin and not due to the presence of C3b or C3(H₂O) captured with properdin, which could have accelerated the formation of convertases and/or increased the signal per se. This was further supported by the data indicating a similar level of function between serum-derived properdin and pure P3 oligomers (FIG. 13A). The outcome of the properdin functional ELISA was compared to the function in a properdin functional hemolytic assay that uses AP-sensitive ER, properdin-depleted sera, and a properdin source (FIG. 15). Unlike the functional ELISA (FIG. 13), the hemolytic assay was not able to consistently detect greater function in higher order oligomers (i.e., Pn>P4>P3>P2), even when the hemolytic assay parameters were varied, including time allotted for complement activation and percentage of properdin depleted serum. Pn resulted in little activity, which has been previously shown to be due to complement consumption in the fluid phase that does not occur for physiological properdin oligomers. The previous study that detected Pn-mediated complement consumption using neuraminidase-treated sheep erythrocytes also detected greater activity in P4 versus P3 and P3 versus P2, unlike the hemolysis assay herein. Differences between the method used may, in part, explain the differences in findings. The functional properdin ELISA does not result in consumption of Pn in the fluid phase because properdin is captured to the solid phase first, then complement proteins (in the context of properdin-depleted sera) are added in a subsequent step to facilitate convertase assembly on the captured properdin, followed by C3b deposition. This contrasts with hemolytic assays in which properdin sources are mixed simultaneously with properdin-depleted sera (complement protein source) and the erythrocytes, permitting complement activation and consumption on properdin aggregates (i.e., Pn) in the fluid phase instead of properdin mediating surface-driven complement-mediated hemolysis.

Given that neutrophils serve important roles at sites of inflammation and release properdin when activated, the functional assay was applied to characterize the oligomeric ratio of neutrophil-derived properdin. The functional assay demonstrated that neutrophil-derived properdin was associated with a 5-fold reduction in C3b deposition compared to donor matched serum-derived properdin (FIG. 17). The cause for this was identified using size exclusion chromatography where the data indicated that neutrophils release properdin predominantly as P2, while P3 and P4 levels are reduced as compared to properdin in NHS (FIGS. 18A-18E). This demonstrates properdin released from neutrophils exist in a ratio distinct from properdin present in healthy serum. In agreement with the notion that properdin-producing cells may secrete properdin at different oligomeric states, a previous study described T-cell-derived properdin as having approximately 100-fold more activity than serum-derived properdin, although the reason for this enhanced activity was never reported. Given that properdin is secreted from numerous sources in addition to neutrophils, these data indicate the ratio of properdin oligomeric forms may differ according to cell type, although whether the release of lower order properdin oligomers is unique to neutrophils remains unknown. In addition, neutrophils release properdin in response to numerous activators including bacterial lipopolysaccharide, tumor necrosis factor-a, interleukin-8, granulocyte macrophage colony stimulating factor, C5a, N-formylmethionine-leucyl-phenylalanine (fMLP), PMA, interferon-a, and influenza A virus. It remains to be determined if neutrophils stimulated by activators other than PMA leads to the release of properdin oligomers at distinct ratios.

The finding that neutrophils release properdin oligomers as mainly P2 is especially relevant in the context of inflammatory microenvironments where changes in the ratio of properdin oligomers may influence AP activity and may thus represent a previously unknown complement regulatory mechanism. Properdin released from neutrophils as predominantly lower order oligomers may be advantageous to maintain a basal, low level AP activity near the site of neutrophil activation in the microenvironment to avoid premature consumption of properdin. This would be due to higher order properdin oligomers, specifically P4, being the first oligomeric form to be utilized by AP convertases and preferentially consumed, given it has the most vertices to interact with convertases and thus the greatest avidity for surfaces on which convertases are formed. Properdin released mainly as P2 may avoid its premature consumption and enable it to eventually assemble to the approximate 1:2:1 ratio in serum and carry out its important functions in circulation, in cellular microenvironments, and at cell-cell interfaces.

In general, protein oligomerization states may be affected by changes in (a) protein concentration; (b) physiological conditions including temperature, pH, ionic strength; or (c) molecular signaling by ligand binding or post-translational modifications. Specifically, properdin oligomerization may be influenced by post-translational modifications such as O-linked glycans present in TSR4. Moreover, denaturation with guanidine converts properdin into an equal mix of P2:P3:P4 (1:1:1) oligomers. Properdin oligomerization is also sensitive to changes in pH. Properdin exposed to acidic, denaturing conditions (pH 2.3) redistributed to a mixture containing similar ratios of all three physiological oligomers and returned to an approximate 1:2:1 ratio of oligomers upon renaturing at pH 7.4. Acidic pH is characteristic of inflammatory microenvironments, such as the tumor microenvironment, synovial fluid of traumatic and osteoarthritic joints, and bacterial biofilms. Interestingly, intercellular pH increases when neutrophils are stimulated with C5a or fMLP that could influence properdin oligomerization. In this example, the pH of the neutrophil supernatant was neutral, indicating it is possible that other factors may influence oligomer state. However, it also indicates that neutrophil oligomer ratio may actually be mostly steady-state and that the combination of different oligomer ratios of properdin derived from different cell sources results in the final approximate 1:2:1 ratio in serum. In agreement with the possibility of consistent ratios, exchange from one oligomer form to another has been reported as slow and that the oligomers are highly stable in vitro.

The molecular mechanisms responsible for the oligomerization ratio of neutrophil-derived properdin and whether, or how, it assimilates to the approximate 1:2:1 ratio in serum, remain unknown. In conclusion, this example utilized an ELISA-based functional assay to describe a previously unknown phenomenon whereby neutrophil-derived properdin was released in an oligomeric ratio (predominantly P2) that was distinct from serum. This knowledge contributes to understanding molecular mechanisms of AP regulation in inflammatory microenvironments.

### Example III—Properdin Functional Assay can be Used to Detect Properdin Function in a Variety of Biological Samples (Beyond Normal Human Serum)

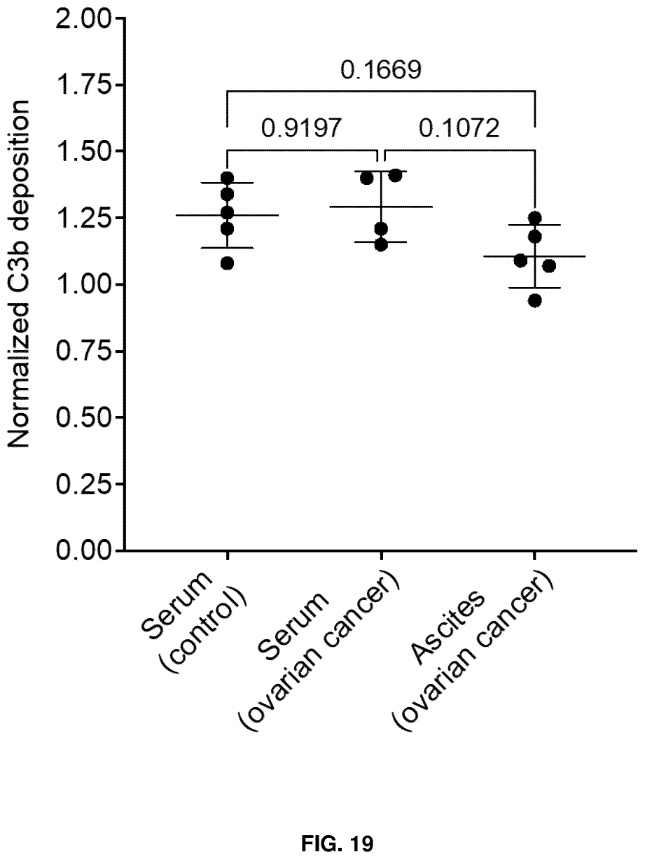

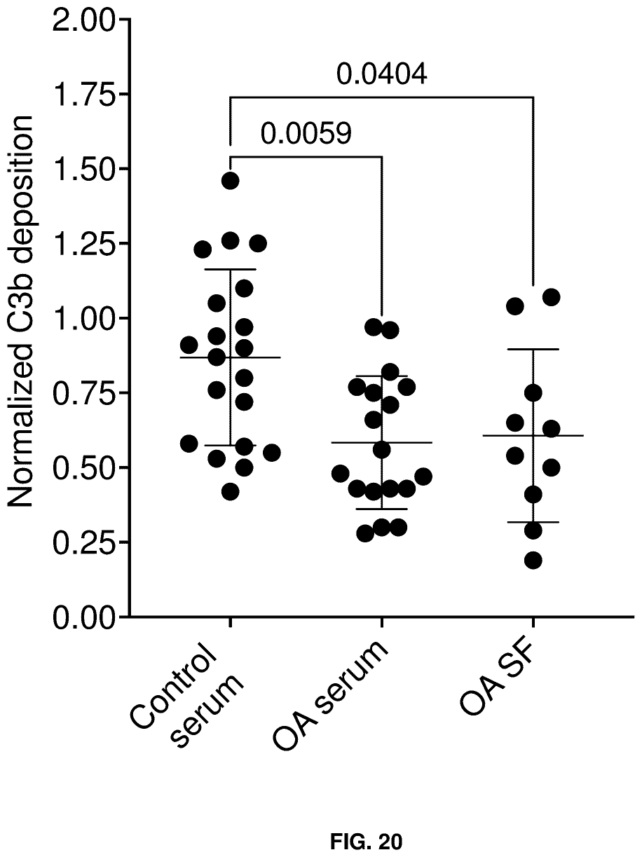

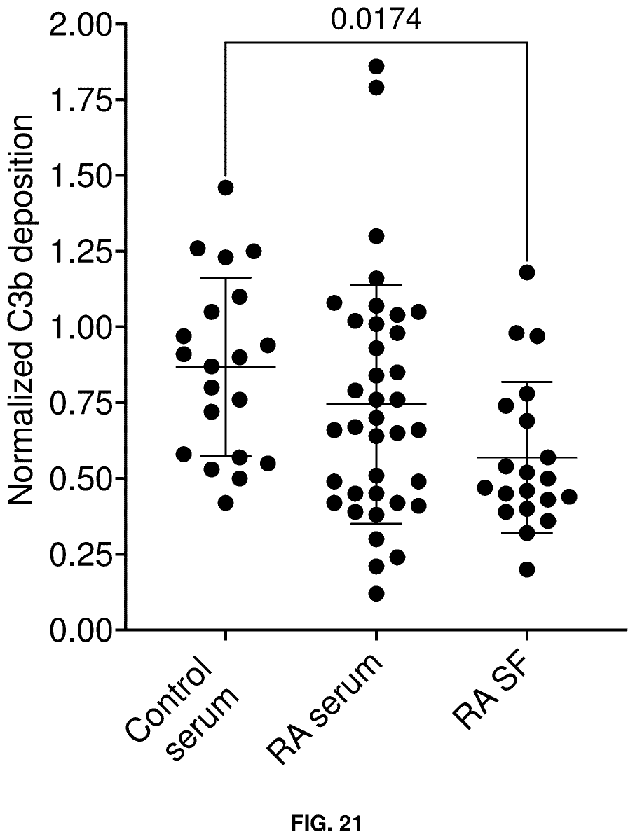

The properdin functional ELISA was used to assess properdin function (high binding, half area plate) in other biological samples, namely, ascitic fluid and synovial fluid. The results are shown in FIGS. 19-21.

A 96-well, high-binding, half-area, ELISA plate was coated with 50 μl/well of 1 μg/ml non-inhibitory anti-properdin monoclonal antibody 6E9E6 diluted in PBS overnight at 4° C. The plate was washed 4× with wash buffer and blocked for 2 h at 37° C. with 125 μl/well blocking buffer in PBS. Properdin sources were diluted to equivalent concentrations within each assay (ng/ml; indicated in figures) in dilution buffer and 20 mM EDTA was then added to the plate and incubated at 37° C. for 1 h for properdin capture by the coating antibody. Background was determined by adding properdin-depleted serum (instead of a properdin source) in dilution buffer. The plate was washed 4× with 130 μl/well wash buffer and while on ice, 50 μl/well properdin-depleted serum diluted 1/10-in GVB with 5 mM MgEGTA added to the plate while cold water was added to empty wells not containing any sample to ensure even temperature distribution across the plate. The plate was incubated for 45 minutes at 37° C. 12.5 μl/well cold 0.1 M EDTA was added to all wells to stop complement activity, and the plate was washed 4× with 130 μl/well wash buffer. C3b deposited covalently on the plate was detected with 50 μl/well biotinylated anti-C3b monoclonal antibody at 200 ng/ml for 1 h at 37° C. The plate was washed 4× with wash buffer and incubated for 45 or 90 min (as indicated in figure legend) at 37° C. with horseradish peroxidase-streptavidin diluted 1:2500 in dilution buffer. The plate was washed 4× with 130 μl/well wash buffer and incubated for 30 min with 10-parts ABTS to 1-part ABTS Enhancer at room temperature and absorbance was read at 405 nM on a Tecan Infinite M200 spectrophotometer. During each incubation, the plate was sealed with a plastic film. Data was normalized by dividing C3b deposition of each sample by C3b deposition of an internal plate control, NHS. This method was used for assessing properdin function in the rheumatoid arthritis cohort.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof.

Therefore, it is intended that the invention not be limited to the particular embodiment disclosed herein contemplated for carrying out this invention, but that the invention will include all embodiments falling within the scope of the claims.

What is claimed is:

1. A method for conducting an enzyme linked immunosorbent assay (ELISA) for detecting the activity of properdin, the method comprising:
i) coating a surface with a non-inhibitory anti-properdin antibody, and incubating for a desired time and at a first desired temperature;
ii) adding a blocking agent to prevent non-specific binding and to block any remaining active sites, and incubating for a second desired time and at a second desired temperature;
iii) adding a biological sample that contains properdin to the surface of step ii), and incubating for a third desired time and at a third desired temperature;
iv) adding properdin-depleted serum to the surface of step iii), wherein the properdin-depleted serum provides complement proteins including C3(H₂O), Factor B and Factor D, and, wherein C3(H₂O) and Factor B bind to properdin, and wherein Factor D cleaves Factor B to form C3(H₂O)Bb that, in turn, cleaves C3 and deposits C3b covalently on the proteins on the surface; and
v) adding an anti-C3b antibody to detect the C3b on the surface.

2. The method of claim 1, wherein the biological sample comprises a neutrophil supernatant.

3. The method of claim 2, wherein the neutrophil supernatant has a neutral pH.

4. The method of claim 1, wherein the biological sample comprises synovial fluid.

5. The method of claim 4, wherein the synovial fluid is from a subject having rheumatoid arthritis.

6. The method of claim 4, wherein the synovial fluid is from a subject having osteoarthritis.

7. The method of claim 1, wherein the biological sample comprises serum.

8. The method of claim 1, wherein the biological sample comprises ascitic fluid.

9. The method of claim 8, wherein the ascitic fluid is from a subject having a cancer.

10. The method of claim 1, wherein the biological sample is added in ethylenediaminetetraacetic acid.

11. The method of claim 1, further comprising removing non-physiological forms of properdin (Pns) from the biological sample by size exclusion chromatography prior to adding the biological sample to the surface of step ii).

12. The method of claim 1, wherein the non-inhibitory anti-properdin antibody comprises a monoclonal antibody.

13. The method of claim 1, wherein the properdin-depleted serum sample is diluted 1/20 or 1/18-fold in 5 mM magnesium ethylene glycol tetraacetic acid (MgEGTA).

14. The method of claim 1, wherein C3b deposition is measured by adding biotinylated anti-C3b antibody and streptavidin-horseradish peroxidase.
